(19) United States
(12) Patent Application Publication
(10) Pub. No.: US 2007/0249535 A1
(43) Pub. Date: Oct. 25, 2007

(54) Methods for treating bone tumors

(76) Inventors: John C. Lee, San Antonio, TX (US); Lee-Chuan C. Yeh, San Antonio, TX (US)

Correspondence Address: FISH & NEAVE IP GROUP;ROPES & GRAY LLP, 1211 AVENUE OF THE AMERICAS, NEW YORK, NY, 10036-8704, US

(21) Appl. No.: 11/715,233

(22) Filed: Mar. 6, 2007

Related U.S. Application Data
(63) Continuation of application No. PCT/US05/32521, filed on Sep. 9, 2005.
(60) Provisional application No. 60/609,196, filed on Sep. 9, 2004.

(51) Int. Cl. A61K 38/00; A61P 35/00

(52) U.S. Cl. 514/12

(57) ABSTRACT

The present invention provides methods of treating bone cancer, inducing differentiation of bone tumor cells, inhibiting bone tumor growth, inducing bone tumor regression or treating a hyperproliferative cell disorder by administering a pharmaceutically effective amount of a bone morphogenic protein or a nucleic acid encoding the bone morphogenic protein.

### FIELD OF THE INVENTION

The present invention relates to methods of treating bone cancer. More particularly, it relates to methods of inducing differentiation of tumor cells into bone.

### BACKGROUND OF THE INVENTION

Although bone cancer is not as prevalent as other forms of cancer, it represents 5 percent of all childhood cancers. There are 5000 new cases of primary bone cancer diagnosed each year in the U.S., approximately one fifth of which are osteosarcomas. Bone cancers can affect any bone in the body. There are two types of bone cancers—primary and secondary. Primary bone cancer refers to cancers which start in the bone, whereas secondary bone cancers refers to cancers which start in other parts of the body, such as breasts, lung, and prostate, and later metastasize to bone.

There are several types of bone cancer, including osteosarcomas, chondrosarcomas and osteocarcinomas. Osteosarcomas (also referred to as osteogenic sarcomas or osteochondrosarcomas) are malignant tumors derived from bone cells. Chondrosarcomas are malignant tumors derived from cartilage cells and form in bone. Osteocarcinomas are metastatic carcinomas in bone.

The exact cause of bone cancer is not known, but it is believed to be due to DNA mutations—either inherited or acquired after birth. Other suggested risk factors, include but are not limited to, teenage growth spurts, being tall for a specific age, previous treatment with radiation for another type of cancer, presence of a benign (non-cancerous) bone disease, presence of certain rare, inherited cancers, such as Li-Fraumeni syndrome and retinoblastoma, lifestyle factors such as high-fat diets, lack of exercise, smoking and alcohol consumption.

Treatment depends on the type of cancer, whether the primary tumor has metastasized, and the size and location of the primary tumor. The main types of therapy used to treat bone cancers include, but are not limited to, surgery, radiotherapy, chemotherapy, amputation (in the case of tumors in limbs) and replacement with bone graft or metal prostheses.

Although there has been considerable progress in developing new regimens for treating bone cancers, such methods still subject patients untoward side effects, such as nausea, vomiting, anemia, hair loss, general malaise, fatigue, lowered resistance to infection, damage to body organs and depression. Therefore, there remains a need for new methods of treating patients suffering from bone cancers.

### SUMMARY OF THE INVENTION

Applicants have solved the above problem by discovering that treatment of tumors with a bone morphogenic protein results in differentiation of the tumor cells into bone. Accordingly, in some embodiments, the invention provides a method of treating bone cancer in a mammal comprising the step of administering to the mammal a pharmaceutically effective amount of a bone morphogenic protein or a nucleic acid encoding the bone morphogenic protein.

In some embodiments, the invention provides a method of inducing differentiation of bone tumor cells in a mammal comprising the step of administering to the mammal a pharmaceutically effective amount of a bone morphogenic protein or a nucleic acid encoding the bone morphogenic protein. In some embodiments, the invention also provides a method or inhibiting bone tumor growth in a mammal comprising the step of administering to the mammal a pharmaceutically effective amount of a bone morphogenic protein or a nucleic acid encoding the bone morphogenic protein. In yet other embodiments, the invention provides a method of inducing bone tumor regression in a mammal comprising the step of administering to the mammal a pharmaceutically effective amount of a bone morphogenic protein or a nucleic acid encoding the bone morphogenic protein.

In some embodiments, the tumor is a sarcoma or a carcinoma. In a preferred embodiment, the sarcoma is an osteosarcoma.

In some embodiments, the invention provides a method of treating a hyperproliferative cell disorder in a mammal comprising the step of administering to the mammal a pharmaceutically effective amount of a bone morphogenic protein or a nucleic acid encoding the bone morphogenic protein. In some embodiments, the hyperproliferative cell is selected from the group consisting of bone, lung and prostate cells.

In some embodiments, the bone morphogenic protein includes, but is not limited to, OP-1, OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17, BMP-18, DPP, Vg1, Vgr, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, NODAL, UNIVIN, SCREW, ADMP, NEURAL, and fragments thereof. In some embodiments, the bone morphogenic protein is selected from the group consisting of OP-1, BMP-5, BMP-6, GDF-5, GDF-6 and GDF-7, CDMP-1, CDMP-2 and CDMP-3. In some embodiments, the bone morphogenic protein is selected from the group consisting of OP-1, BMP-5 and BMP-6. Preferably, the bone morphogenic protein is OP-1.

In some embodiments, the bone morphogenic protein comprises an amino acid sequence having at least 70% homology with the C-terminal 102-106 amino acids, including the conserved seven cysteine domain, of human OP-1.

In some embodiments, the nucleic acid used in the invention is a viral vector comprising a gene that encodes a bone morphogenic protein, and wherein the viral vector, includes but not limited to, an adenoviral vector, a lentiviral vector, a baculoviral vector, an Epstein Barr viral vector, a papovaviral vector, a vaccinia viral vector and a herpes simplex viral vector. In a preferred embodiment, the viral vector is an adenoviral vector, a baculoviral vector or a lentiviral vector. More preferably, the viral vector is an adenoviral vector.

In some embodiments, the invention provides a bone morphogenic protein or nucleic acid formulated as a gel, an aqueous solution, a paste or a putty. In some embodiments, the formulation is a sustained release formulation. In some embodiments, the bone morphogenic protein or nucleic acid encoding it is formulated for local administration.

### BRIEF DESCRIPTION OF THE DRAWINGS

FIG. 1 shows the effect of OP-1 on cell morphology and cell counts (viable and total cell counts) in the osteosarcoma cell line SaOS-2. Cells were treated with either 0.5 μg/ml or 100 μof OP-1 for 24 h. Cell morphology was monitored using an inverted microscope equipped with a CCD camera. Viable and total cell counts (value in parentheses) were determined using the trypan blue exclusion assay.

FIG. 2 shows the effect of OP-1 on cell morphology and cell counts (viable and total cell counts) in the osteosarcoma cell line MG-63. Cells were treated with either 0.5 μg/ml or 100 μg/ml of OP-1 for 24 h. Cell morphology was monitored using an inverted microscope equipped with a CCD camera. Viable and total cell counts (value in parentheses) were determined using the trypan blue exclusion assay.

FIG. 3 shows the effect of OP-1 on cell morphology and cell counts (viable and total cell counts) in the lung carcinoma cell line A549. Cells were treated with either 0.5 μg/ml or 100 μg/ml of OP-1 for 24 h. Cell morphology was monitored using an inverted microscope equipped with a CCD camera. Viable and total cell counts (value in parentheses) were determined using the trypan blue exclusion assay.

FIG. 4 shows the mRNA expression of the BMP receptor ActR-I in the MG-63, A-549, PC-3 and SaOS-2 cell lines. Values were normalized to the 18S rRNA control (=1 ) and expressed as normalized intensity ×10,000.

FIG. 5 shows the mRNA expression of the BMP receptor BMPR-IA in the MG-63, A-549, PC-3 and SaOS-2 cell lines. Values were normalized to the 18S rRNA control (=1) and expressed as normalized intensity ×10,000.

FIG. 6 shows the mRNA expression of the BMP receptor BMPR-IB in the MG-63, A-549, PC-3 and SaOS-2 cell lines. Values were normalized to the 18S rRNA control (=1) and expressed as normalized intensity ×10,000.

FIG. 7 shows the mRNA expression of the BMP receptor BMPR-II in the MG-63, A-549, PC-3 and SaOS-2 cell lines. Values were normalized to the 18S rRNA control (=1) and expressed as normalized intensity ×10,000.

FIG. 8 shows the effect of OP-1 treatment on SaOS-2 cell proliferation. Treatment of SaOS-2 cells with OP-1 increased cell proliferation in a dose-dependent manner, as measured by the tetrazolium colorimetric assay (CellTiter96AQ Cell Proliferation Assay) and monitoring color development at 490 nm. Values were normalized to control untreated cells (=1).

FIG. 9 shows the effect of OP-1 treatment on MG-63 cell proliferation, as measured by thymidine incorporation. Values were normalized to control untreated cells (=1).

FIG. 10 shows the effect of OP-1 treatment on A549 cell proliferation, as measured by thymidine incorporation. Values were normalized to control untreated cells (=1).

FIG. 11 shows the effect of OP-1 treatment on PC-3 cell proliferation, as measured by thymidine incorporation. Values were normalized to control untreated cells (=1).

FIG. 12 shows the effect of OP-1 treatment on the alkaline phosphatase activity in SaOS-2 cells. Values are normalized to solvent-treated control cells (=1).

FIG. 13 shows the effect of OP-1 treatment on the alkaline phosphatase activity in MG-63 cells. Values are normalized to solvent-treated control cells (=1).

FIG. 14 shows the in vivo mass growth of MG-63 cells in nude mice injected with control or OP-1-treated cells as a function of post-injection time. Each experiment included n=8 for both the control and OP-1 -treated groups. Control mice were also observed for 49 days and no growth/tumors were detected.

FIG. 15 shows the in vivo mass growth of SaOS-2 cells in nude mice injected with control or OP-1-treated cells as a function of post-injection time. Each experiment included n=8 for both the control and OP-1-treated groups. Control mice were also observed for 49 days and no growth/tumors were detected.

FIG. 16 shows the in vivo mass growth of A549 cells in nude mice injected with control or OP-1-treated cells as a function of post-injection time. Each experiment included n=8 for both the control and OP- 1-treated groups.

FIG. 17 shows the in vivo mass growth of PC-3 cells in nude mice injected with control or OP-1-treated cells as a function of post-injection time. Each experiment included n=8 for both the control and OP-1-treated groups.

FIG. 18 shows the histological appearance of a subcutaneous bone nodule containing bone marrow in a nude mouse injected with MG-63 cells treated with OP-1. The histologic image is represented at a magnification of 200×.

FIG. 19 shows that several tumor cell foci were present within the bone nodule in a nude mouse injected with OP-1-treated MG-63 osteosarcoma cells. The histologic image is represented at a magnification of 100×.

FIG. 20A shows the histologic appearance of a subcutaneous mass comprised mainly of bone, bone marrow and focus of tumor cells in a nude mouse injected with OP-1-treated SaOS-02 osteosarcoma cells. The histologic image is represented at a magnification of 100×.

FIG. 20B shows a higher magnification of the tumor mass shown in FIG. 18A. There is a distinct tumor cell bone marrow interface. The histologic image is represented at a magnification of 200×.

FIG. 21 shows the histologic appearance of a subcutaneous mass comprised mainly of bone, bone marrow and focus of tumor cells in a nude mouse injected with OP-1-treated SaOS-02 osteosarcoma cells. The histologic image is represented at a magnification of 200×.

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

In order to further define the invention, the following terms and definitions are provided herein.

The term “pharmaceutically effective amount” refers to an amount effective to repair, regenerate, promote, accelerate, prevent degradation, or form bone. A “pharmaceutically effective amount” also means the amount required to improve the clinical symptoms of a patient. The therapeutic methods or methods of treating cancer described herein are not to be interpreted or otherwise limited to “curing” cancer. Rather, the therapeutic methods and methods of treating cancer are intended to mean effecting a beneficial and/or desirable alteration in the general health of a patient suffering from cancer (e.g., bone cancer, lung cancer or prostate cancer). A skilled healthcare practitioner would recognize that such benefits and/or desirable effect include, but are not limited to tumor regression (decrease in tumor size), a decrease in metastasis, improved vital functions of the patient, improved well-being of the patient, decrease in pain, improved appetite, improvement in the patient's weight and any combination thereof.

The term “patient” refers to an animal including a mammal (e.g., a human).

The term “pharmaceutically acceptable carrier or adjuvant” refers to a non-toxic carrier or adjuvant that may be administered to a patient, together with a morphogenic protein of this invention, and which does not destroy the pharmacological activity thereof.

The term “bone morphogenic protein (BMP)” refers to a protein belonging to the BMP family of the TGF-β superfamily of proteins (BMP family) based on DNA and amino acid sequence homology. A protein belongs to the BMP family according to this invention when it has at least 50% amino acid sequence identity with at least one known BMP family member within the conserved C-terminal cysteine-rich domain, which characterizes the BMP protein family. Preferably, the protein has at least 70% amino acid sequence identity with at least one known BMP family member within the conserved C-terminal cysteine rich domain. Members of the BMP family may have less than 50% DNA or amino acid sequence identity overall.

The term “amino acid sequence homology” is understood to include both amino acid sequence identity and similarity. Homologous sequences share identical and/or similar amino acid residues, where similar residues are conservative substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence. Certain particularly preferred morphogenic polypeptides share at least 60%, and preferably 70% amino acid sequence identity with the C-terminal 102-106 amino acids, defining the conserved seven-cysteine domain of human OP-1 and related proteins.

Amino acid sequence homology can be determined by methods well known in the art. For instance, to determine the percent homology of a candidate amino acid sequence to the sequence of the seven-cysteine domain, the two sequences are first aligned. The alignment can be made with, e.g., the dynamic programming algorithm described in Needleman et al., J. Mol. Biol., 48, pp. 443 (1970), and the Align Program, a commercial software package produced by DNAstar, Inc. The teachings by both sources are incorporated by reference herein. An initial alignment can be refined by comparison to a multi-sequence alignment of a family of related proteins. Once the alignment is made and refined, a percent homology score is calculated. The aligned amino acid residues of the two sequences are compared sequentially for their similarity to each other. Similarity factors include similar size, shape and electrical charge. One particularly preferred method of determining amino acid similarities is the PAM250 matrix described in Dayhoff et al., Atlas of Protein Sequence and Structure, 5, pp. 345-352 (1978 & Supp.), which is incorporated herein by reference. A similarity score is first calculated as the sum of the aligned pair wise amino acid similarity scores. Insertions and deletions are ignored for the purposes of percent homology and identity. Accordingly, gap penalties are not used in this calculation. The raw score is then normalized by dividing it by the geometric mean of the scores of the candidate sequence and the seven-cysteine domain. The geometric mean is the square root of the product of these scores. The normalized raw score is the percent homology.

The term “conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., supra. Examples of conservative substitutions are substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. The term “conservative variant” or “conservative variation” also includes the use of a substituting amino acid residue in place of an amino acid residue in a given parent amino acid sequence, where antibodies specific for the parent sequence are also specific for, i.e., “cross-react” or “immuno-react” with, the resulting substituted polypeptide sequence.

The term “osteogenic protein (OP)” refers to a morphogenic protein that is capable of inducing a progenitor cell to form cartilage and/or bone. The bone may be intramembranous bone or endochondral bone. Most osteogenic proteins are members of the BMP protein family and are thus also BMPs. As described elsewhere herein, the class of proteins is typified by human osteogenic protein (hOP-1). Other osteogenic proteins useful in the practice of the invention include osteogenically active forms of OP-1, OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17, BMP-18, DPP, Vg1, Vgr-1, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, UNIVIN, NODAL, SCREW, ADMP or NEURAL, and amino acid sequence variants thereof. Osteogenic proteins suitable for use with applicants' invention can be identified by means of routine experimentation using the art-recognized bioassay described by Reddi and Sampath (Sampath et al., Proc. Natl. Acad. Sci., 84, pp. 7109-13, incorporated herein by reference).

Methods and Compositions for Tumor Differentiation and Inhibition of Tumor Growth

The bone morphogenic proteins and nucleic acids encoding the bone morphogenic proteins of this invention may be used for treating bone cancer. In some embodiments, a method of inducing differentiation of bone tumors using a bone morphogenic protein or a nucleic acid encoding the bone morphogenic protein is provided. In some embodiments, the invention also provides a method of inhibiting bone tumor growth in a mammal using a bone morphogenic protein or a nucleic acid encoding the bone morphogenic protein. In yet other embodiments, the invention provides a method of inducing bone tumor regression in a mammal using a bone morphogenic protein or a nucleic acid encoding the bone morphogenic protein. In some embodiments, the invention provides a method of treating a hyperproliferative cell disorder using a bone morphogenic protein or a nucleic acid encoding the bone morphogenic protein. The bone tumors according to this invention may be sarcomas or carcinomas. Preferably, the sarcoma is an osteosarcoma.

In some embodiments, the bone cancer is a primary bone cancer (i.e., cancer which starts in the bone). In other embodiments, the bone cancer is a secondary bone cancer (i.e., the cancer starts in another part of the body and metastasizes to the bone).

Bone Morphogenic Protein Family

The BMP family, named for its representative bone morphogenic/osteogenic protein family members, belongs to the TGF-β protein superfamily. Of the reported “BMPs” (BMP-1 to BMP-18), isolated primarily based on sequence homology, all but BMP-1 remain classified as members of the BMP family of morphogenic proteins (Ozkaynak et al., EMBO J., 9, pp. 2085-93 (1990)).

The BMP family includes other structurally-related members which are morphogenic proteins, including the drosophila decapentaplegic gene complex (DPP) products, the Vg1 product of Xenopus laevis and its murine homolog, Vgr-1 (see, e.g., Massagué, Annu. Rev. Cell Biol., 6, pp. 597-641 (1990), incorporated herein by reference).

The C-terminal domains of BMP-3, BMP-5, BMP-6, and OP-1 (BMP-7) are about 60% identical to that of BMP-2, and the C-terminal domains of BMP-6 and OP-1 are 87% identical. BMP-6 is likely the human homolog of the murine Vgr-1 (Lyons et al., Proc. Natl. Acad. Sci. U.S.A., 86, pp. 4554-59 (1989)); the two proteins are 92% identical overall at the amino acid sequence level (U.S. Pat. No. 5,459,047, incorporated herein by reference). BMP-6 is 58% identical to the Xenopus Vg-1 product.

The naturally occurring bone morphogenic proteins share substantial amino acid sequence homology in their C-terminal regions (domains). Typically, the naturally occurring osteogenic proteins are translated as a precursor, having an N-terminal signal peptide sequence typically less than about 30 residues, followed by a “pro” domain that is cleaved to yield the mature C-terminal domain of approximately 97-106 amino acids. The signal peptide is cleaved rapidly upon translation, at a cleavage site that can be predicted in a given sequence using the method of Von Heijne Nucleic Acids Research, 14, pp. 4683-4691 (1986). The pro domain typically is about three times larger than the fully processed mature C-terminal domain.

Another characteristic of the BMP protein family members is their apparent ability to dimerize. Several bone-derived osteogenic proteins (OPs) and BMPs are found as homo- and heterodimers in their active forms. The ability of OPs and BMPs to form heterodimers may confer additional or altered morphogenic inductive capabilities on bone morphogenic proteins. Heterodimers may exhibit qualitatively or quantitatively different binding affinities than homodimers for OP and BMP receptor molecules. Altered binding affinities may in turn lead to differential activation of receptors that mediate different signaling pathways, which may ultimately lead to different biological activities or outcomes. Altered binding affinities could also be manifested in a tissue or cell type-specific manner, thereby inducing only particular progenitor cell types to undergo proliferation and/or differentiation.

In preferred embodiments, the pair of osteogenic polypeptides have amino acid sequences each comprising a sequence that shares a defined relationship with an amino acid sequence of a reference bone morphogenic protein. Herein, preferred osteogenic polypeptides share a defined relationship with a sequence present in osteogenically active human OP-1, SEQ ID NO: 1. However, any one or more of the naturally occurring or biosynthetic sequences disclosed herein similarly could be used as a reference sequence. Preferred osteogenic polypeptides share a defined relationship with at least the C-terminal six cysteine domain of human OP-1, residues 335-431 of SEQ ID NO: 1. Preferably, osteogenic polypeptides share a defined relationship with at least the C-terminal seven cysteine domain of human OP-1, residues 330-431 of SEQ ID NO: 1. That is, preferred polypeptides in a dimeric protein with bone morphogenic activity each comprise a sequence that corresponds to a reference sequence or is functionally equivalent thereto.

Functionally equivalent sequences include functionally equivalent arrangements of cysteine residues disposed within the reference sequence, including amino acid insertions or deletions which alter the linear arrangement of these cysteines, but do not materially impair their relationship in the folded structure of the dimeric bone morphogenic protein, including their ability to form such intra- or inter-chain disulfide bonds as may be necessary for morphogenic activity. Functionally equivalent sequences further include those wherein one or more amino acid residues differs from the corresponding residue of a reference sequence, e.g., the C-terminal seven cysteine domain (also referred to herein as the conserved seven cysteine skeleton) of human OP-1, provided that this difference does not destroy bone morphogenic activity. Accordingly, conservative substitutions of corresponding amino acids in the reference sequence are preferred. Amino acid residues that are conservative substitutions for corresponding residues in a reference sequence are those that are physically or functionally similar to the corresponding reference residues, e.g., that have similar size, shape, electric charge, chemical properties including the ability to form covalent or hydrogen bonds, or the like. Particularly preferred conservative substitutions are those fulfilling the criteria defined for an accepted point mutation in Dayhoff et al., supra, the teachings of which are incorporated by reference herein.

The osteogenic protein OP-1 has been described (see, e.g., Oppermann et al., U.S. Pat. No. 5,354,557, incorporated herein by reference). Natural-sourced osteogenic protein in its mature, native form is a glycosylated dimer typically having an apparent molecular weight of about 30-36 kDa as determined by SDS-PAGE. When reduced, the 30 kDa protein gives rise to two glycosylated peptide subunits having apparent molecular weights of about 16 kDa and 18 kDa. In the reduced state, the protein has no detectable osteogenic activity. The unglycosylated protein, which also has osteogenic activity, has an apparent molecular weight of about 27 kDa. When reduced, the 27 kDa protein gives rise to two unglycosylated polypeptides, having molecular weights of about 14 kDa to 16 kDa, capable of inducing endochondral bone formation in a mammal. Osteogenic proteins may include forms having varying glycosylation patterns, varying N-termini, and active truncated or mutated forms of native protein. As described above, particularly useful sequences include those comprising the C-terminal 96 or 102 amino acid sequences of DPP (from Drosophila), Vg1 (from Xenopus), Vgr-1 (from mouse), the OP-1 and OP-2 proteins,(see U.S. Pat. No. 5,011,691 and Oppermann et al., incorporated herein by reference), as well as the proteins referred to as BMP-2, BMP-3, BMP-4 (see WO88/00205, U.S. Pat. No. 5,013,649 and WO91/18098, incorporated herein by reference), BMP-5 and BMP-6 (see WO90/11366, PCT/US90/01630, incorporated herein by reference), BMP-8 and BMP-9.

Preferred osteogenic proteins of this invention comprise at least one polypeptide including, but not limited to OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, BMP-12, BMP-13, BMP-14, BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, CDMP-1, CDMP-2, CDMP-3, dorsalin-1, DPP, Vg-1, Vgr-1, 60A protein, NODAL, UNIVIN, SCREW, ADMP, NEURAL, TGF-β, amino acid sequence variants and homologs thereof, including species homologs, thereof and fragments thereof. Preferably, the osteogenic protein comprises at least one polypeptide selected from OP-1 (BMP-7), BMP-2, BMP-4, BMP-5, BMP-6, GDF-5, GDF-6, GDF-7, CDMP-1, CDMP-2 or CDMP-3; more preferably, OP-1 (BMP-7), BMP-5, BMP-6, GDF-5, GDF-6, GDF-7, CDMP-1, CDMP-2 or CDMP-3; even more preferably, OP-1 (BMP-7), BMP-5 or BMP-6; and most preferably, OP-1 (BMP-7).

Publications disclosing these sequences, as well as their chemical and physical properties, include: OP-1 and OP-2 (U.S. Pat. No. 5,011,691; U.S. Pat. No. 5,266,683; Ozkaynak et al., EMBO J., 9, pp. 2085-2093 (1990); OP-3 (WO94/10203 (PCT US93/10520)); BMP-2, BMP-3, BMP-4, (WO88/00205; Wozney et al. Science, 242, pp. 1528-1534 (1988)); BMP-5 and BMP-6, (Celeste et al., PNAS, 87, 9843-9847 (1991)); Vgr-1 (Lyons et al., PNAS, 86, pp. 4554-4558 (1989)); DPP (Padgett et al. Nature, 325, pp. 81-84 (1987)); Vg-1 (Weeks, Cell, 51, pp. 861-867 (1987)); BMP-9 (WO95/33830 (PCT/US95/07084); BMP-10 (WO94/26893 (PCT/US94/05290); BMP-11 (WO94/26892 (PCT/US94/05288); BMP-12 (WO95/16035 (PCT/US94/14030); BMP-13 (WO95/16035 (PCT/US94/14030); GDF-1 (WO92/00382 (PCT/US91/04096) and Lee et al. PNAS, 88, pp. 4250-4254 (1991); GDF-8 (WO94/21681 (PCT/US94/03019); GDF-9 (WO94/15966 (PCT/US94/00685); GDF-10 (WO95/10539 (PCT/US94/11440); GDF-11 (WO96/01845 (PCT/US95/08543); BMP-15 (WO96/36710 (PCT/US96/06540); MP-121 (WO96/01316 (PCT/EP95/02552); GDF-5 (CDMP-1, MP52) (WO94/15949 (PCT/US94/00657) and WO96/14335 (PCT/US94/12814) and WO93/16099 (PCT/EP93/00350)); GDF-6 (CDMP-2, BMP13) (WO95/01801 (PCT/US94/07762) and WO96/14335 and WO95/10635 (PCT/US94/14030)); GDF-7 (CDMP-3, BMP12) (WO95/10802 (PCT/US94/07799) and WO95/10635 (PCT/US94/14030)); BMP-17 and BMP-18 (U.S. Pat. No. 6,027,917). The above publications are incorporated herein by reference.

In another embodiment, useful proteins include biologically active biosynthetic constructs, including novel biosynthetic bone morphogenic proteins and chimeric proteins designed using sequences from two or more known bone morphogenic proteins.

In another embodiment of this invention, a bone morphogenic protein or osteogenic protein may be prepared synthetically to induce tissue formation. Bone morphogenic proteins prepared synthetically may be native, or may be non-native proteins, i.e., those not otherwise found in nature.

Non-native osteogenic proteins have been synthesized using a series of consensus DNA sequences (U.S. Pat. No. 5,324,819, incorporated herein by reference). These consensus sequences were designed based on partial amino acid sequence data obtained from natural osteogenic products and on their observed homologies with other genes reported in the literature having a presumed or demonstrated developmental function.

Several of the biosynthetic consensus sequences (called consensus osteogenic proteins or “COPs”) have been expressed as fusion proteins in prokaryotes (see, e.g., U.S. Pat. No. 5,011,691, incorporated herein by reference. These include COP-1, COP-3, COP-4, COP-5, COP-7 and COP-16, as well as other proteins known in the art. Purified fusion proteins may be cleaved, refolded, implanted in an established animal model and shown to have bone- and/or cartilage-inducing activity. The currently preferred synthetic osteogenic proteins comprise two synthetic amino acid sequences designated COP-5 (SEQ. ID NO: 2) and COP-7 (SEQ. ID NO: 3).

Oppermann et al., U.S. Pat. Nos. 5,011,691 and 5,324,819, which are incorporated herein by reference, describe the amino acid sequences of COP-5 and COP-7 as shown below:

| | |
|---|---|
| COP5 | LYVDFS-DVGWDDWIVAPPGYQAFYCHGECPFPLAD |
| COP7 | LYVDFS-DVGWNDWIVAPPGYHAFYCHGECPFPLAD |
| COP5 | HFNSTN--H-AVVQTLVNSVNSKI--PKACCVPTELSA |
| COP7 | HLNSTN--H-AVVQTLVNSVNSKI--PKACCVPTELSA |
| COP5 | ISMLYLDENEKVVLKYNQEMVVEGCGCR |
| COP7 | ISMLYLDENEKVVLKYNQEMVVEGCGCR |

In these amino acid sequences, the dashes (—) are used as fillers only to line up comparable sequences in related proteins. Differences between the aligned amino acid sequences are highlighted.

The DNA and amino acid sequences of these and other BMP family members are published and may be used by those of skill in the art to determine whether a newly identified protein belongs to the BMP family. New BMP-related gene products are expected by analogy to possess at least one bone morphogenic activity and thus classified as a BMP.

In one preferred embodiment of this invention, the bone morphogenic protein comprises a pair of subunits disulfide bonded to produce a dimeric species, wherein at least one of the subunits comprises a recombinant peptide belonging to the BMP protein family. In another preferred embodiment of this invention, the bone morphogenic protein comprises a pair of subunits that produce a dimeric species formed through non-covalent interactions, wherein at least one of the subunits comprises a recombinant peptide belonging to the BMP protein family. Non-covalent interactions include Van der Waals, hydrogen bond, hydrophobic and electrostatic interactions. The dimeric species may be a homodimer or heterodimer and is capable of inducing cell proliferation and/or tissue formation. In other preferred embodiments, the bone morphogenic protein is a monomer.

In certain preferred embodiments, bone morphogenic proteins useful herein include those in which the amino acid sequences comprise a sequence sharing at least 70% amino acid sequence homology or “similarity”, and preferably 75%, 80%, 85%, 90%, 95%, or 98% homology or similarity, with a reference bone morphogenic protein selected from the foregoing naturally occurring proteins. Preferably, the reference protein is human OP-1, and the reference sequence thereof is the C-terminal seven cysteine domain present in osteogenically active forms of human OP-1, residues 330-431 of SEQ ID NO: 1. In certain embodiments, a polypeptide suspected of being functionally equivalent to a reference bone morphogenic polypeptide is aligned therewith using the method of Needleman, et al., supra, implemented conveniently by computer programs such as the Align program (DNAstar, Inc.). As noted above, internal gaps and amino acid insertions in the candidate sequence are ignored for purposes of calculating the defined relationship, conventionally expressed as a level of amino acid sequence homology or identity, between the candidate and reference sequences. “Amino acid sequence homology” is understood herein to include both amino acid sequence identity and similarity. Homologous sequences share identical and/or similar amino acid residues, where similar residues are conservation substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence. In a currently preferred embodiment, the reference sequence is OP-1. Bone morphogenic proteins useful herein accordingly include allelic, phylogenetic counterpart and other variants of the preferred reference sequence, whether naturally-occurring or biosynthetically produced (e.g., including “muteins” or “mutant proteins”), as well as novel members of the general morphogenic family of proteins, including those set forth and identified above. Certain particularly preferred bone morphogenic polypeptides share at least 60% amino acid identity with the preferred reference sequence of human OP-1, still more preferably at least 65%, 70%, 75%, 80%, 85%, 90%, 95%, or 98% amino acid identity therewith.

In another embodiment, useful osteogenic proteins include those sharing the conserved seven cysteine domain and sharing at least 70% amino acid sequence homology (similarity) within the C-terminal active domain, as defined herein. In still another embodiment, the osteogenic proteins of the invention can be defined as osteogenically active proteins having any one of the generic sequences defined herein, including OPX (SEQ ID NO: 4) and Generic Sequences 7 (SEQ ID NO: 5) and 8 (SEQ ID NO: 6), or Generic Sequences 9 (SEQ ID NO: 7) and 10 (SEQ ID NO: 8).

The family of bone morphogenic polypeptides useful in the present invention, and members thereof, can be defined by a generic amino acid sequence. For example, Generic Sequence 7 (SEQ ID NO: 5) and Generic Sequence 8 (SEQ ID NO: 6 ) are 96 and 102 amino acid sequences, respectively, and accommodate the homologies shared among preferred protein family members identified to date, including at least OP-1, OP-2, OP-3, CBMP-2A, CBMP-2B, BMP-3, 60A, DPP, Vg1, BMP-6, Vgr-1, and GDF-1. The amino acid sequences for these proteins are described herein and/or in the art, as summarized above. The generic sequences include both the amino acid identity shared by these sequences in the C-terminal domain, defined by the six and seven cysteine skeletons (Generic Sequences 7 and 8 , respectively), as well as alternative residues for the variable positions within the sequence. The generic sequences provide an appropriate cysteine skeleton where inter- or intramolecular disulfide bonds can form, and contain certain critical amino acids likely to influence the tertiary structure of the folded proteins. In addition, the generic sequences allow for an additional cysteine at position 36 (Generic Sequence 7) or position 41 (Generic Sequence 8), thereby encompassing the morphogenically active sequences of OP-2 and OP-3.

Generic Sequence 7

```
                        Leu Xaa Xaa Xaa Phe Xaa Xaa
                        1 5
Xaa Gly Trp Xaa Xaa Xaa Xaa Xaa Xaa Pro
                10 15
Xaa Xaa Xaa Xaa Ala Xaa Tyr Cys Xaa Gly
                20 25
Xaa Cys Xaa Xaa Pro Xaa Xaa Xaa Xaa Xaa
                30 35
Xaa Xaa Xaa Asn His Ala Xaa Xaa Xaa Xaa
                40 45
Xaa Xaa Xaa Xaa Xaa Xaa Xaa Xaa Xaa Xaa
                50 55
Xaa Xaa Xaa Cys Cys Xaa Pro Xaa Xaa Xaa
                60 65
Xaa Xaa Xaa Xaa Xaa Leu Xaa Xaa Xaa Xaa
                70 75
Xaa Xaa Xaa Val Xaa Leu Xaa Xaa Xaa Xaa
                80 85
Xaa Met Xaa Val Xaa Xaa Cys Xaa Cys Xaa
                90 95
```

wherein each independently is selected from a group of one or more specified amino acids defined as follows: “res.” means “residue” and Xaa at res.2=(Tyr or Lys); Xaa at res.3=Val or Ile); Xaa at res.4=(Ser, Asp or Glu); Xaa at res. 6=(Arg, Gln, Ser, Lys or Ala); Xaa at res.7=(Asp or Glu); Xaa at res.8=(Leu, Val or Ile); Xaa at res.11=(Gln, Leu, Asp, His, Asn or Ser); Xaa at res.12=(Asp, Arg, Asn or Glu); Xaa at res.13=(Trp or Ser); Xaa at res. 14=(Ile or Val); Xaa at res. 15=(Ile or Val); Xaa at res. 16 (Ala or Ser); Xaa at res. 18=(Glu, Gln, Leu, Lys, Pro or Arg); Xaa at res.19=(Gly or Ser); Xaa at res.20=(Tyr or Phe); Xaa at res.21=(Ala, Ser, Asp, Met, His, Gln, Leu or Gly); Xaa at res.23=(Tyr, Asn or Phe); Xaa at res.26=(Glu, His, Tyr, Asp, Gln, Ala or Ser); Xaa at res.28=(Glu, Lys, Asp, Gln or Ala); Xaa at res.30=(Ala, Ser, Pro, Gln, Ile or Asn); Xaa at res.31=(Phe, Leu or Tyr); Xaa at res.33=(Leu, Val or Met); Xaa at res. 34=(Asn, Asp, Ala, Thr or Pro); Xaa at res.35=(Ser, Asp, Glu, Leu, Ala or Lys); Xaa at res.36=(Tyr, Cys, His, Ser or Ile); Xaa at res.37=(Met, Phe, Gly or Leu); Xaa at res.38=(Asn, Ser or Lys); Xaa at res.39=(Ala, Ser, Gly or Pro); Xaa at res. 40=(Thr, Leu or Ser); Xaa at res.44=(Ile, Val or Thr); Xaa at res.45=(Val, Leu, Met or Ile); Xaa at res.46=(Gln or Arg); Xaa at res.47=(Thr, Ala or Ser); Xaa at res.48=(Leu or Ile); Xaa at res.49=(Val or Met); Xaa at res.50=(His, Asn or Arg); Xaa at res.51=(Phe, Leu, Asn, Ser, Ala or Val); Xaa at res.52=(Ile, Met, Asn, Ala, Val, Gly or Leu); Xaa at res.53=(Asn, Lys, Ala, Glu, Gly or Phe); Xaa at res.54=(Pro, Ser or Val); Xaa at res.55=(Glu, Asp, Asn, Gly, Val, Pro or Lys); Xaa at res.56=(Thr, Ala, Val, Lys, Asp, Tyr, Ser, Gly, Ile or His); Xaa at res. 57=(Val, Ala or Ile); Xaa at res.58=(Pro or Asp); Xaa at res.59=(Lys, Leu or Glu); Xaa at res.60=(Pro, Val or Ala); Xaa at res.63=(Ala or Val); Xaa at res. 65=(Thr, Ala or Glu); Xaa at res.66=(Gln, Lys, Arg or Glu); Xaa at res.67=(Leu, Met or Val); Xaa at res.68=(Asn, Ser, Asp or Gly); Xaa at res.69=(Ala, Pro or Ser); Xaa at res.70=(Ile, Thr, Val or Leu); Xaa at res.71=(Ser, Ala or Pro); Xaa at res.72=(Val, Leu, Met or Ile); Xaa at res.74=(Tyr or Phe); Xaa at res. 75=(Phe, Tyr, Leu or His); Xaa at res.76=(Asp, Asn or Leu); Xaa at res.77=(Asp, Glu, Asn, Arg or Ser); Xaa at res.78=(Ser, Gln, Asn, Tyr or Asp); Xaa at res. 79=(Ser, Asn, Asp, Glu or Lys); Xaa at res.80=(Asn, Thr or Lys); Xaa at res. 82=(Ile, Val or Asn); Xaa at res.84=(Lys or Arg); Xaa at res.85=(Lys, Asn, Gln, His, Arg or Val); Xaa at res.86=(Tyr, Glu or His); Xaa at res.87=(Arg, Gln, Glu or Pro); Xaa at res.88=(Asn, Glu, Trp or Asp); Xaa at res.90=(Val, Thr, Ala or Ile); Xaa at res.92=(Arg, Lys, Val, Asp, Gln or Glu); Xaa at res.93=(Ala, Gly, Glu or Ser); Xaa at res.95=(Gly or Ala) and Xaa at res.97=(His or Arg).

Generic Sequence 8 (SEQ ID NO: 6) includes all of Generic Sequence 7 and in addition includes the following sequence (SEQ ID NO: 9) at its N-terminus:

SEQ ID NO: 9

Cys Xaa Xaa Xaa Xaa
1 5

Accordingly, beginning with residue 7, each “Xaa” in Generic Sequence 8 is a specified amino acid defined as for Generic Sequence 7, with the distinction that each residue number described for Generic Sequence 7 is shifted by five in Generic Sequence 8. Thus, “Xaa at res.2=(Tyr or Lys)” in Generic Sequence 7 refers to Xaa at res. 7 in Generic Sequence 8. In Generic Sequence 8, Xaa at res.2=(Lys, Arg, Ala or Gln); Xaa at res.3=(Lys, Arg or Met); Xaa at res.4=(His, Arg or Gln); and Xaa at res. 5=(Glu, Ser, His, Gly, Arg, Pro, Thr, or Tyr).

In another embodiment, useful osteogenic proteins include those defined by Generic Sequences 9 and 10, defined as follows.

Specifically, Generic Sequences 9 and 10 are composite amino acid sequences of the following proteins: human OP-1, human OP-2, human OP-3, human BMP-2, human BMP-3, human BMP-4, human BMP-5, human BMP-6, human BMP-8, human BMP-9, human BMP 10, human BMP-11, Drosophila 60A, Xenopus Vg-1, sea urchin UNIVIN, human CDMP-1 (mouse GDF-5), human CDMP-2 (mouse GDF-6, human BMP-13), human CDMP-3 (mouse GDF-7, human BMP-12), mouse GDF-3, human GDF-1, mouse GDF-1, chicken DORSALIN, dpp, Drosophila SCREW, mouse NODAL, mouse GDF-8, human GDF-8, mouse GDF-9, mouse GDF-10, human GDF-11, mouse GDF-11, human BMP-15, and rat BMP3b. Like Generic Sequence 7, Generic Sequence 9 is a 96 amino acid sequence that accommodates the C-terminal six cysteine skeleton and, like Generic Sequence 8, Generic Sequence 10 is a 102 amino acid sequence which accommodates the seven cysteine skeleton.

Generic Sequence 9

```
Xaa Xaa Xaa Xaa Xaa Xaa Xaa Xaa Xaa Xaa
1 5 10
Xaa Xaa Xaa Xaa Xaa Xaa Pro Xaa Xaa Xaa
                                15 20
Xaa Xaa Xaa Xaa Cys Xaa Gly Xaa Cys Xaa
                                25 30
Xaa Xaa Xaa Xaa Xaa Xaa Xaa Xaa Xaa Xaa
                                35 40
Xaa Xaa Xaa Xaa Xaa Xaa Xaa Xaa Xaa Xaa
                                45 50
Xaa Xaa Xaa Xaa Xaa Xaa Xaa Xaa Xaa Xaa
                                55 60
Xaa Cys Xaa Pro Xaa Xaa Xaa Xaa Xaa Xaa
                                65 70
Xaa Xaa Leu Xaa Xaa Xaa Xaa Xaa Xaa Xaa
                                75 80
Xaa Xaa Xaa Xaa Xaa Xaa Xaa Xaa Xaa Xaa
                                85 90
Xaa Xaa Xaa Cys Xaa Cys Xaa
                                95
```

wherein each Xaa is independently selected from a group of one or more specified amino acids defined as follows: “res.” means “residue” and Xaa at res. 1 =(Phe, Leu of Glu); Xaa at res. 2=(Tyr, Phe, His, Arg, Thr, Lys, Gln, Val or Glu); Xaa at res. 3=(Val, Ile, Leu, or Asp); Xaa at res. 4=(Ser, Asp, Glu, Asn or Phe); Xaa at res. 5=(Phe or Glu); Xaa at res. 6=(Arg, Gln, Lys, Ser, Glu, Ala or Asn); Xaa at res. 7=(Asp, Glu, Leu, Ala or Gln); Xaa at res. 8=(Leu, Val, Met, Ile or Phe); Xaa at res. 9=(Gly, His or Lys); Xaa at res. 10=(Trp or Met); Xaa at res. 11=(Gln, Leu, His, Glu, Asn, Asp, Ser or Gly); Xaa at res. 12=(Asp, Asn, Ser, Lys, Arg, Glu or His); Xaa at res. 13=(Trp or Ser); Xaa at res. 14=(Ile or Val); Xaa at res.15=(Ile of Val); Xaa at res. 16=(Ala, Ser, Tyr or Trp); Xaa at res. 18=(Glu, Lys, Gln, Met, Pro, Leu, Arg, His or Lys); Xaa at res. 19=(Gly, Glu, Asp, Lys, Ser, Gln, Arg or Phe); Xaa at res. 20=(Tyr or Phe); Xaa at res. 21=(Ala, Ser, Gly, Met, Gln, His, Glu, Asp, Leu, Asn, Lys, or Thr); Xaa at res. 22=(Ala or Pro); Xaa at res.23=(tyr, Phe, Asn, Ala or Arg); Xaa at res. 24=(Tyr, His, Glu, Phe or Arg); Xaa at res. 26=(Glu, Asp, Ala, Ser, Tyr, His, Lys, Arg, Gln or Gly); Xaa at res. 28=(Glu, Asp, Leu, Val, Lys, Gly , Thr, Ala or Gln); Xaa at res. 30=(Ala, Ser, Ile, Asn, Pro, Glu, Asp, Phe, Gln or Leu); Xaa at res. 31=(Phe, Tyr, Leu, Asn, Gly or Arg); Xaa at res. 32=(Pro, Ser, Ala or Val); Xaa at res. 33=(Leu, Met, Glu, Phe or Val); Xaa at res. 34=(Asn, Asp, Thr, Gly, Ala, Arg, Leu or Pro); Xaa at res. 35=(Ser, Ala, Glu, Asp, Thr, Leu, Lys, Gln or His); Xaa at res. 36=(Tyr, His, Cys, Ile, Arg, Asp, Asn, Lys, Ser, Glu or Gly); Xaa at res. 37=(Met, Leu, Phe, Val, Gly or Tyr); Xaa at res. 38=(Asn, Glu, Thr, Pro, Lys, His, Gly, Met, Val or Arg); Xaa at res. 39=(Ala, Ser, Gly, Pro or Phe); Xaa at res. 40=(Thr, Ser, Leu, Pro, His or Met); Xaa at res. 41=(Asn, Lys, Val, Thr or Gln); Xaa at res. 42=(His, Tyr or Lys); Xaa at res. 43=(Ala, Thr, Leu or Tyr); Xaa at res. 44=(Ile, Thr, Val, Phe, Tyr, Met or Pro); Xaa at res. 45=(Val, Leu, Met, Ile or His); Xaa at res. 46=(Gln, Arg or Thr); Xaa at res. 47=(Thr, Ser, Ala, Asn or His); Xaa at res. 48=(Leu, Asn or Ile); Xaa at res. 49=(Val, Met, Leu, Pro or Ile); Xaa at res. 50=(His, Asn, Arg, Lys, Tyr or Gln); Xaa at res. 51=(Phe, Leu, Ser, Asn, Met, Ala, Arg, Glu, Gly or Gln); Xaa at res. 52=(Ile, Met, Leu, Val, Lys, Gln, Ala or Tyr); Xaa at res. 53=(Asn, Phe, Lys, Glu, Asp, Ala, Gln, Gly, Leu or Val); Xaa at res. 54=(Pro, Asn, Ser, Val or Asp); Xaa at res. 55=(Glu, Asp, Asn, Lys, Arg, Ser, Gly, Thr, Gln, Pro or His); Xaa at res. 56=(Thr, His, Tyr, Ala, Ile, Lys, Asp, Ser, Gly or Arg); Xaa at res. 57=(Val, Ile, Thr, Ala, Leu or Ser); Xaa at res. 58=(Pro, Gly, Ser, Asp or Ala); Xaa at res. 59=(Lys, Leu, Pro, Ala, Ser, Glu, Arg or Gly); Xaa at res. 60=(Pro, Ala, Val, Thr or Ser); Xaa at res. 61=(Cys, Val or Ser); Xaa at res. 63=(Ala, Val or Thr); Xaa at res. 65=(Thr, Ala, Glu, Val, Gly, Asp or Tyr); Xaa at res. 66=(Gln, Lys, Glu, Arg or Val); Xaa at res. 67=(Leu, Met, Thr or Tyr); Xaa at res. 68=(Asn, Ser, Gly, Thr, Asp, Glu, Lys or Val); Xaa at res. 69=(Ala, Pro, Gly or Ser); Xaa at res. 70=(Ile, Thr, Leu or Val); Xaa at res. 71=(Ser, Pro, Ala, Thr, Asn or Gly); Xaa at res. 2=(Val, Ile, Leu or Met); Xaa at res. 74=(Tyr, Phe, Arg, Thr, Tyr or Met); Xaa at res. 75=(Phe, Tyr, His, Leu, Ile, Lys, Gln or Val); Xaa at res. 76=(Asp, Leu, Asn or Glu); Xaa at res. 77=(Asp, Ser, Arg, Asn, Glu, Ala, Lys, Gly or Pro); Xaa at res. 78=(Ser, Asn, Asp, Tyr, Ala, Gly, Gln, Met, Glu, Asn or Lys); Xaa at res. 79=(Ser, Asn, Glu, Asp, Val, Lys, Gly, Gln or Arg); Xaa at res. 80=(Asn, Lys, Thr, Pro, Val, Ile, Arg, Ser or Gln); Xaa at res. 81=(Val, Ile, Thr or Ala); Xaa at res. 82=(Ile, Asn, Val, Leu, Tyr, Asp or Ala); Xaa at res. 83=(Leu, Tyr, Lys or Ile); Xaa at res. 84=(Lys, Arg, Asn, Tyr, Phe, Thr, Glu or Gly); Xaa at res. 85=(Lys, Arg, His, Gln, Asn, Glu or Val); Xaa at res. 86=(Tyr, His, Glu or Ile); Xaa at res. 87=(Arg, Glu, Gln, Pro or Lys); Xaa at res. 88=(Asn, Asp, Ala, Glu, Gly or Lys); Xaa at res. 89=(Met or Ala); Xaa at res. 90=(Val, Ile, Ala, Thr, Ser or Lys); Xaa at res. 91=(Val or Ala); Xaa at res. 92=(Arg, Lys, Gln, Asp, Glu, Val, Ala, Ser or Thr); Xaa at res. 93=(Ala, Ser, Glu, Gly, Arg or Thr); Xaa at res. 95=(Gly, Ala or Thr); Xaa at res. 97=(His, Arg, Gly, Leu or Ser). Further, after res. 53 in rBMP3b and mGDF-10 there is an Ile; after res. 54 in GDF-1 there is a T; after res. 54 in BMP3 there is a V; after res. 78 in BMP-8 and Dorsalin there is a G; after res. 37 in hGDF-1 there is Pro, Gly, Gly, Pro.

Generic Sequence 10 (SEQ ID NO: 8) includes all of Generic Sequence 9 (SEQ ID NO: 7) and in addition includes the following sequence (SEQ ID NO: 9) at its N-terminus:

SEQ ID NO: 9

Cys Xaa Xaa Xaa Xaa
1 5

Accordingly, beginning with residue 6, each “Xaa” in Generic Sequence 10 is a specified amino acid defined as for Generic Sequence 9, with the distinction that each residue number described for Generic Sequence 9 is shifted by five in Generic Sequence 10. Thus, “Xaa at res. 1=(Tyr, Phe, His, Arg, Thr, Lys, Gln, Val or Glu)” in Generic Sequence 9 refers to Xaa at res. 6 in Generic Sequence 10. In Generic Sequence 10, Xaa at res. 2=(Lys, Arg, Gln, Ser, His, Glu, Ala, or Cys); Xaa at res. 3=(Lys, Arg, Met, Lys, Thr, Leu, Tyr, or Ala); Xaa at res. 4=(His, Gin, Arg, Lys, Thr, Leu, Val, Pro, or Tyr); and Xaa at res. 5=(Gln, Thr, His, Arg, Pro, Ser, Ala, Gln, Asn, Tyr, Lys, Asp, or Leu).

As noted above, certain currently preferred bone morphogenic polypeptide sequences useful in this invention have greater than 60% identity, preferably greater than 65%, 70%, 75%, 80%, 85%, 90%, 95%, or 98% identity, with the amino acid sequence defining the preferred reference sequence of hOP-1. These particularly preferred sequences include allelic and phylogenetic counterpart variants of the OP-1 and OP-2 proteins, including the Drosophila 60A protein. Accordingly, in certain particularly preferred embodiments, useful bone morphogenic proteins include active proteins comprising pairs of polypeptide chains within the generic amino acid sequence herein referred to as “OPX” (SEQ ID NO: 4), which defines the seven cysteine skeleton and accommodates the homologies between several identified variants of OP-1 and OP-2. As described therein, each Xaa at a given position independently is selected from the residues occurring at the corresponding position in the C-terminal sequence of mouse or human OP-1 or OP-2.

```
Cys Xaa Xaa His Glu Leu Tyr Val Ser Phe Xaa Asp
1 5 10
Leu Gly Trp Xaa Asp Trp Xaa Ile Ala Pro Xaa Gly
                15 20
Tyr Xaa Ala Tyr Tyr Cys Glu Gly Glu Cys Xaa Phe
25 30 35
Pro Leu Xaa Ser Xaa Met Asn Ala Thr Asn His Ala
                        40 45
Ile Xaa Gln Xaa Leu Val His Xaa Xaa Xaa Pro Xaa
        50 55 60
Xaa Val Pro Lys Xaa Cys Cys Ala Pro Thr Xaa Leu
                                65 70
Xaa Ala Xaa Ser Val Leu Tyr Xaa Asp Xaa Ser Xaa
                75 80
Asn Val Ile Leu Xaa Lys Xaa Arg Asn Met Val Val
85 90 95
Xaa Ala Cys Gly Cys His
                        100
```

wherein Xaa at res. 2=(Lys or Arg); Xaa at res. 3=(Lys or Arg); Xaa at res. 11=(Arg or Gln); Xaa at res. 16=(Gln or Leu); Xaa at res. 19=(Ile or Val); Xaa at res. 23=(Glu or Gln); Xaa at res. 26=(Ala or Ser); Xaa at res. 35=(Ala or Ser); Xaa at res. 39=(Asn or Asp); Xaa at res. 41=(Tyr or Cys); Xaa at res. 50=(Val or Leu); Xaa at res. 52=(Ser or Thr); Xaa at res. 56=(Phe or Leu); Xaa at res. 57=(Ile or Met); Xaa at res. 58=(Asn or Lys); Xaa at res. 60=(Glu, Asp or Asn); Xaa at res. 61=(Thr, Ala or Val); Xaa at res. 65=(Pro or Ala); Xaa at res. 71=(Gln or Lys); Xaa at res. 73=(Asn or Ser); Xaa at res. 75=(Ile or Thr); Xaa at res. 80=(Phe or Tyr); Xaa at res. 82=(Asp or Ser); Xaa at res. 84=(Ser or Asn); Xaa at res. 89=(Lys or Arg); Xaa at res. 91=(Tyr or His); and Xaa at res. 97=(Arg or Lys).

In still another preferred embodiment, useful osteogenically active proteins have polypeptide chains with amino acid sequences comprising a sequence encoded by a nucleic acid that hybridizes, under low, medium or high stringency hybridization conditions, to DNA or RNA encoding reference bone morphogenic sequences, e.g., C-terminal sequences defining the conserved seven cysteine domains of OP-1, OP-2, BMP-2, BMP-4, BMP-5, BMP-6, 60A, GDF-3, GDF-6, GDF-7 and the like. As used herein, high stringent hybridization conditions are defined as hybridization according to known techniques in 40% formamide, 5×SSPE, 5× Denhardt's Solution, and 0.1% SDS at 37° C. overnight, and washing in 0.1×SSPE, 0.1% SDS at 50° C. Standard stringent conditions are well characterized in commercially available, standard molecular cloning texts. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984): Nucleic Acid Hybridization (B. D. Hames & S. J. Higgins eds. 1984); and B. Perbal, A Practical Guide To Molecular Cloning (1984), the disclosures of which are incorporated herein by reference.

As noted above, proteins useful in the present invention generally are dimeric proteins comprising a folded pair of the above polypeptides. Such bone morphogenic proteins are inactive when reduced, but are active as oxidized homodimers and when oxidized in combination with others of this invention to produce heterodimers. Thus, members of a folded pair of bone morphogenic polypeptides in a morphogenically active protein can be selected independently from any of the specific polypeptides mentioned above. In some embodiments, the bone morphogenic protein is a monomer.

The bone morphogenic proteins useful in the materials and methods of this invention include proteins comprising any of the polypeptide chains described above, whether isolated from naturally-occurring sources, or produced by recombinant DNA or other synthetic techniques, and includes allelic and phylogenetic counterpart variants of these proteins, as well as muteins thereof, fragments thereof and various truncated and fusion constructs. Deletion or addition mutants also are envisioned to be active, including those which may alter the conserved C-terminal six or seven cysteine domain, provided that the alteration does not functionally disrupt the relationship of these cysteines in the folded structure. Accordingly, such active forms are considered the equivalent of the specifically described constructs disclosed herein. The proteins may include forms having varying glycosylation patterns, varying N-termini, a family of related proteins having regions of amino acid sequence homology, and active truncated or mutated forms of native or biosynthetic proteins, produced by expression of recombinant DNA in host cells.

The bone morphogenic proteins contemplated herein can be expressed from intact or truncated cDNA or from synthetic DNAs in prokaryotic or eukaryotic host cells, and purified, cleaved, refolded, and dimerized to form morphogenically active compositions. Currently preferred host cells include, without limitation, prokaryotes including E. coli or eukaryotes including yeast, or mammalian cells, such as CHO, COS or BSC cells. One of ordinary skill in the art will appreciate that other host cells can be used to advantage. Detailed descriptions of the bone morphogenic proteins useful in the practice of this invention, including how to make, use and test them for osteogenic activity, are disclosed in numerous publications, including U.S. Pat. Nos. 5,266,683 and 5,011,691, the disclosures of which are incorporated by reference herein, as well as in any of the publications recited herein, the disclosures of which are incorporated herein by reference.

Thus, in view of this disclosure and the knowledge available in the art, skilled genetic engineers can isolate genes from cDNA or genomic libraries of various different biological species, which encode appropriate amino acid sequences, or construct DNAs from oligonucleotides, and then can express them in various types of host cells, including both prokaryotes and eukaryotes, to produce large quantities of active proteins capable of stimulating bone and cartilage morphogenesis in a mammal. In addition, the skilled worker can also prepare nucleic acid molecules which express the proteins of this invention in vivo.

In some embodiments, the bone morphogenic protein includes, but is not limited to OP-1, OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17, BMP-18, DPP, Vg1, Vgr, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, NODAL, UNIVIN, SCREW, ADMP, NEURAL, and amino acid sequence variants thereof. In some embodiments, the bone morphogenic protein comprises an amino acid sequence having at least 70% homology with the C-terminal 102-106 amino acids, including the conserved seven cysteine domain, of human OP-1, said bone morphogenic protein being capable of inducing repair of bone and/or cartilage defects.

In a preferred embodiment, the bone morphogenic protein is OP-1, BMP-5, BMP-6, GDF-5, GDF-6 and GDF-7, CDMP-1, CDMP-2 or CDMP-3. In a more preferred embodiment, the bone morphogenic protein is OP-1, BMP-5 or BMP-6. In a most preferred embodiment, the bone morphogenic protein is OP-1.

Gene Therapy

As used herein, the term “transformation” or “transform” refers to any genetic modification of cells and includes both “transfection” and “transduction”.

As used herein, “transfection of cells” refers to the acquisition by a cell of new genetic material by incorporation of added DNA. Thus, transfection refers to the insertion of nucleic acid (e.g., DNA) into a cell using physical or chemical methods. Several transfection techniques are known to those of ordinary skill in the art including: calcium phosphate DNA co-precipitation (Methods in Molecular Biology, Vol. 7, Gene Transfer and Expression Protocols, Ed. E. J. Murray, Humana Press (1991)); DEAE-dextran (supra); electroporation (supra); cationic liposome-mediated transfection (supra); and tungsten particle-facilitated microparticle bombardment (Johnston, S. A., Nature 346: 776-777 (1990)); and strontium phosphate DNA co-precipitation (Brash D. E. et al. Molec. Cell. Biol. 7: 2031-2034 (1987). Each of these methods is well represented in the art.

As used herein, “transduction of cells” refers to the process of transferring nucleic acid into a cell using a DNA or RNA virus. One or more isolated polynucleotide sequences encoding one or more interferon proteins contained within the virus may be incorporated into the chromosome of the transduced cell. Alternatively, a cell is transduced with a virus but the cell will not have the isolated polynucleotide incorporated into its chromosomes but will be capable of expressing interferon extrachromosomally within the cell.

According to one embodiment of the invention, the cells are transformed (i.e., genetically modified) ex vivo. The cells are isolated from a mammal (preferably a human) and transformed (i.e., transduced or transfected in vitro) with a vector containing an isolated bone morphogenic polynucleotide gene operatively linked to one or more expression control sequences for expressing a recombinant protein. The cells are then administered to a mammalian recipient for delivery of the bone morphogenic protein in situ. Preferably, the mammalian recipient is a human and the cells to be modified are autologous cells, i.e., the cells are isolated from the mammalian recipient.

According to another embodiment, the cells are transformed or otherwise genetically modified in vivo. The cells from the mammalian recipient (preferably a human), are transformed (i.e., transduced or transfected) in vivo with a vector containing isolated polynucleotide such as a recombinant gene operatively linked to one or more expression control sequences for expressing a bone morphogenic protein and the protein is delivered in situ.

The isolated polynucleotides encoding the bone morphogenic protein is introduced into the cell ex vivo or in vivo by genetic transfer methods, such as transfection or transduction, to provide a genetically modified cell. Various expression vectors (i.e., vehicles for facilitating delivery of the isolated polynucleotide into a target cell) are known to one of ordinary skill in the art.

Typically, the introduced genetic material includes an isolated polynucleotide such as bone morphogenic protein gene (usually in the form of a cDNA) together with a promoter to control transcription of the new gene. The promoter characteristically has a specific nucleotide sequence necessary to initiate transcription. Optionally, the genetic material could include intronic sequences, which will be removed from the mature transcript by RNA splicing. A polyadenylation signal should be present at the 3′ end of the gene to be expressed. The introduced genetic material also may include an appropriate secretion “signal” sequence for secreting the bone morphogenic protein from the cell to the extracellular milieu.

Optionally, the isolated genetic material further includes additional sequences (i.e., enhancers) required to obtain the desired gene transcription activity. For the purpose of this discussion an “enhancer” is simply any non-translated DNA sequence which works contiguous with the coding sequence (in cis) to change the basal transcription level dictated by the promoter. Preferably, the isolated genetic material is introduced into the cell genome immediately downstream from the promoter so that the promoter and coding sequence are operatively linked so as to permit transcription of the coding sequence. Preferred viral expression vectors includes an exogenous promoter element to control transcription of the inserted bone morphogenic protein gene. Such exogenous promoters include both constitutive and inducible promoters.

Naturally-occurring constitutive promoters control the expression of proteins that regulate essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88: 4626-4630 (1991)), adenosine deaminase, phosphoglycerol kinase (PGK), pyruvate kinase, phosphoglycerol mutase, the .beta.-actin promoter (Lai et al., Proc. Natl. Acad. Sci. USA 86: 10006-10010 (1989)), and other constitutive promoters known to those of skill in the art.

In addition, many viral promoters function constitutively in eucaryotic cells. These include: the early and late promoters of SV40 (See Bemoist and Chambon, Nature, 290:304 (1981)); the long terminal repeats (LTRs) of Moloney Leukemia Virus and other retroviruses (See Weiss et al., RNA Tumor Viruses, Cold Spring Harbor Laboratory, Cold Spring Harbor, N.Y. (1985)); the thymidine kinase promoter of Herpes Simplex Virus (HSV) (See Wagner et al., Proc. Nat. Acad. Sci. USA, 78: 1441(1981)); the cytomegalovirus immediate-early (IE1) promoter (See Karasuyama et al., J. Exp. Med., 169: 13 (1989); the promoter of the Rous sarcoma virus (RSV) (Yamamoto et al., Cell, 22:787 (1980)); the adenovirus major late promoter (Yamada et al., Proc. Nat. Acad. Sci. USA, 82: 3567 (1985)), among many others. Accordingly, any of the above-referenced constitutive promoters can be used to control transcription of a gene insert. In some embodiments, tissue specific promoters may be used to deliver the gene to a specific tissue. In other embodiments, tumor specific promoters may be used (e.g., PSA promoter prostate carcinoma).

Selection and optimization of these factors for delivery of a pharmaceutically effective amount of a particular bone morphogenic protein is within the scope of one of ordinary skill in the art.

Any of the methods known in the art for the insertion of polynucleotide sequences into a vector may be used. See, for example, Sambrook et al., Molecular Cloning: A Laboratory Manual, Cold Spring Harbor Laboratory, Cold Spring Harbor, N.Y. (1989) and Ausubel et al., Current Protocols in Molecular Biology, J. Wiley & Sons, NY (1992). Conventional vectors consist of appropriate transcriptional/translational control signals operably bone morphogenic protein.

Expression vectors compatible with mammalian host cells for use in gene therapy of tumor cells include, for example, plasmids; avian, murine and human retroviral vectors; adenovirus vectors; herpes viral vectors; parvoviruses; and non-replicative pox viruses. In particular, replication-defective recombinant viruses can be generated in packaging cell lines that produce only replication-defective viruses. See Current Protocols in Molecular Biology: Sections 9.10-9.14 (Ausubel et al., eds.), Greene Publishing Associates, 1989.

Specific viral vectors for use in gene transfer systems are now well established. See, e.g., Madzak et al., J. Gen. Virol., 73: 1533-36 (1992) (papovavirus SV40); Berkner et al., Curr. Top. Microbiol. Immunol., 158: 39-61 (1992) (adenovirus); Moss et al., Curr. Top. Microbiol. Immunol., 158: 25-38 (1992) (vaccinia virus); Muzyczka, Curr. Top. Microbiol. Immunol., 158: 97-123 (1992) (adeno-associated virus); Margulskee, Curr. Top. Microbiol. Immunol., 158: 67-93 (1992) (herpes simplex virus (HSV) and Epstein-Barr virus (HBV)); Miller, Curr. Top. Microbiol. Immunol., 158: 1-24 (1992) (retrovirus); Brandyopadhyay et al., Mol. Cell. Biol., 4: 749-754 (1984) (retrovirus); Miller et al., Nature, 357: 455-450 (1992) (retrovirus); Anderson, Science, 256: 808-813 (1992) (retrovirus).

Preferred vectors are DNA viruses that include adenoviruses (preferably Ad-2 or Ad-5 based vectors), herpes viruses (preferably herpes simplex virus based vectors), and parvoviruses (preferably “defective” or non-autonomous parvovirus based vectors, more preferably adeno-associated virus based vectors, most preferably AAV-2 based vectors). See, e.g., Ali et al., Gene Therapy 1: 367-384, 1994; U.S. Pat. Nos. 4,797,368 and 5,399,346.

Pharmaceutical Compositions

The bone morphogenic proteins of this invention may be formulated into compositions having a variety of forms. The compositions of this invention will be administered at an effective dose to induce the particular type of tissue at the treatment site selected according to the particular clinical condition addressed. Determination of a preferred pharmaceutical formulation and a pharmaceutically and therapeutically efficient dose regiment for a given application is well within the skill of the art taking into consideration, for example, the administration mode, the condition and weight of the patient, the extent of desired treatment and the tolerance of the patient for the treatment.

Doses expected to be suitable starting points for optimizing treatment regiments are based on the results of in vitro assays, and ex vivo or in vivo assays. Based on the results of such assays, a range of suitable bone morphogenic protein concentrations can be selected to test at a treatment site in animals and then in humans.

The pharmaceutical compositions comprising a bone morphogenic protein this invention may be in a variety of forms. These include, for example, solid, semi-solid and liquid dosage forms such as tablets, pills, powders, liquid solutions or suspensions, suppositories, and injectable and infusible solutions. The preferred form depends on the intended mode of administration and therapeutic application and may be selected by one skilled in the art. Modes of administration may include systemic such as oral, parenteral (such as subcutaneous, intravenous, intraarterial, intralesional, intraosseous, intramuscular, intradermal, transdermal, transmucosal and inhalational), intraperitoneal, topical or local administration. The compositions may be formulated in dosage forms appropriate for each route of administration. In some embodiments, the bone morphogenic proteins of this invention will be administered into the tumor. In other embodiments, the bone morphogenic proteins of this invention will be administered in the vicinity of the tumor. In other embodiments the bone morphogenic proteins of this invention will be administered locally into the tumor.

The pharmaceutical compositions comprising the bone morphogenic proteins may, for example, be placed into sterile, isotonic formulations with or without cofactors which stimulate uptake or stability. The formulation is preferably liquid, or may be lyophilized powder. For example, the bone morphogenic protein may be diluted with a formulation buffer. The solution can be lyophilized, stored under refrigeration and reconstituted prior to administration with sterile Water-For-Injection (USP).

The compositions also will preferably include conventional pharmaceutically acceptable carriers well known in the art (see for example Remington's Pharmaceutical Sciences, 16th Edition, 1980, Mac Publishing Company). Such pharmaceutically acceptable carriers may include other medicinal agents, carriers, genetic carriers, adjuvants, excipients, etc., such as human serum albumin or plasma preparations. The compositions are preferably in the form of a unit dose and will usually be administered as a dose regiment that depends on the particular tissue treatment.

Preferably, the carrier is isotonic with the blood or body fluids of the patient. Examples of such carrier vehicles include water, saline, Ringer's solution, a buffered solution, hyaluronan and dextrose solution. Non-aqueous vehicles such as fixed oils and ethyl oleate are also useful herein. The compositions are preferably in the form of a unit dose and will usually be administered as a dose regimen that depends on the particular tissue treatment.

In some embodiments, the bone morphogenic protein is formulated as a sustained release formulation. There are numerous sustained or slow delivery materials available for preparing the compositions of this invention. They include, but are not limited to, microspheres of polylactic/polyglycolic acid polymers, liposomes, collagen, hyaluronic acid/fibrin matrices, hyaluronic acid, fibrin, chitosan, gelatin, SABER™ System (sucrose acetate isobutyrate (SAIB)), DURIN™ (biodegradabale polymer for drug loaded implants), MICRODUR™ (biodegradable polymers/microencapsulation) and DUROS™ (mini-osmotic pump).

The bone morphogenic proteins of this invention may be dispersed in a biocompatible carrier material that functions as a suitable delivery system for the compounds. Suitable examples of sustained release carriers include semipermeable polymer matrices. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers, 22, pp. 547-56 (1985)); poly(2-hydroxyethyl-methacrylate), ethylene vinyl acetate (Langer et al., J. Biomed. Mater. Res., 15, pp. 167-277 (1981); Langer, Chem. Tech., 12, pp. 98-105 (1982)) or poly-D-(−)-3hydroxybutyric acid (EP 133,988), polylactic acid, poly glycolic acid or polymers of the above.

The bone morphogenic protein of this invention may also be administered using, for example, microspheres, liposomes, other microparticulate delivery systems or sustained release formulations placed in, near, or otherwise in communication with affected tissues, the fluids bathing those tissues or bloodstream bathing those tissues.

Liposomes containing a bone morphogenic protein of this invention can be prepared by well-known methods (See, e.g. DE 3,218,121; Epstein et al., Proc. Natl. Acad. Sci. U.S.A., 82, pp. 3688-92 (1985); Hwang et al., Proc. Natl. Acad. Sci. U.S.A., 77, pp. 4030-34 (1980); U.S. Pat. Nos. 4,485,045 and 4,544,545). Ordinarily the liposomes are of the small (about 200-800 Angstroms) unilamellar type in which the lipid content is greater than about 30 mol. % cholesterol. The proportion of cholesterol is selected to control the optimal rate of bone morphogenic protein release.

The bone morphogenic proteins of this invention may also be attached to liposomes containing other biologically active molecules such as immunosuppressive agents, cytokines, etc., to modulate the rate and characteristics of tissue induction. Attachment of bone morphogenic proteins to liposomes may be accomplished by any known cross-linking agent such as heterobifunctional cross-linking agents that have been widely used to couple toxins or chemotherapeutic agents to antibodies for targeted delivery. Conjugation to liposomes can also be accomplished using the carbohydrate-directed cross-linking reagent 4-(4-maleimidophenyl) butyric acid hydrazide (MPBH) (Duzgunes et al., J. Cell. Biochem. Abst. Suppl. 16E 77 (1992)).

One skilled in the art may create a biocompatible, and or biodegradable formulation of choice for use in the methods of this invention.

A successful carrier for a bone morphogenic protein should perform several important functions. It should act as a delivery system of the bone morphogenic protein and protect the bone morphogenic protein from non-specific proteolysis.

In addition, selected materials must be biocompatible in vivo and preferably biodegradable. Polylactic acid (PLA), polyglycolic acid (PGA), and various combinations have different dissolution rates in vivo.

The carrier may also take the form of a hydrogel. When the carrier material comprises a hydrogel, it refers to a three dimensional network of cross-linked hydrophilic polymers in the form of a gel substantially composed of water, preferably but not limited to gels being greater than 90% water. Hydrogel can carry a net positive or net negative charge, or may be neutral. A typical net negative charged hydrogel is alginate. Hydrogels carrying a net positive charge may be typified by extracellular matrix components such as collagen and laminin. Examples of commercially available extracellular matrix components include Matrigel™ and Vitrogen™. An example of a net neutral hydrogel is highly crosslinked polyethylene oxide, or polyvinyalcohol.

Various growth factors, cytokines, hormones, trophic agents and therapeutic compositions including antibiotics and chemotherapeutic agents, enzymes, enzyme inhibitors and other bioactive agents also may be adsorbed onto or dispersed within the carrier material comprising the bone morphogenic protein, and will also be released over time and slowly absorbed.

Dosage levels of between about 1 μg and about 1000 μg per day, preferably between 3 μg and 50 μg per day of the bone morphogenic protein are useful. As the skilled artisan will appreciate, lower or higher doses than those recited may be required. Specific dosage and treatment regimens for any particular patient will depend upon a variety of factors, including the activity of the specific bone morphogenic protein employed, the age, body weight, general health status, sex, diet, time of administration, rate of excretion, drug combination, the severity of the tissue damage and the judgment of the treating physician.

The bone morphogenic protein of this invention may also be dispersed in an implantable biocompatible carrier material that functions as a suitable delivery or support system for the compounds. Suitable examples of carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and ethyl-L-glutamate (Sidman et al., Biopolymers, 22, pp. 547-56 (1985)); poly(2-hydroxyethyl-methacrylate) or ethylene vinyl acetate (Langer et al., J. Biomed. Mater. Res., 15, pp. 167-277 (1981); Langer, Chem. Tech., 12, pp. 98-105 (1982)). In some embodiments, the carriers are sustained release carriers.

In one embodiment of this invention, the carrier comprises a biocompatible matrix made up of particles or porous materials. The pores are preferably of a dimension to permit progenitor cell migration and subsequent differentiation and proliferation. Various matrices known in the art can be employed (see, e.g., U.S. Pat. Nos. 4,975,526; 5,162,114; 5,171,574 and WO 91/18558, which are herein incorporated by reference).

The particle size should be within the range of 70 μm-850 μm, preferably 70 μm-420 μm, most preferably 150 μm-420 μm. The matrix may be fabricated by close packing particulate material into a shape spanning the particular tissue defect to be treated. Alternatively, a material that is biocompatible, and preferably biodegradable in vivo may be structured to serve as a temporary scaffold and substratum for recruitment of migratory progenitor cells, and as a base for their subsequent anchoring and proliferation.

Useful matrix materials comprise, for example, collagen; homopolymers or copolymers of glycolic acid, lactic acid, and butyric acid, including derivatives thereof; and ceramics, such as hydroxyapatite, tricalcium phosphate and other calcium phosphates. Various combinations of these or other suitable matrix materials also may be useful as determined by the assays set forth herein.

In some embodiments, carriers include particulate, demineralized, guanidine-extracted, species-specific (allogenic) bone, and specially treated particulate, protein-extracted, demineralized xenogenic bone. Optionally, such xenogenic bone powder matrices also may be treated with proteases such as trypsin. Preferably, the xenogenic matrices are treated with one or more fibril modifying agents to increase the intraparticle intrusion volume (porosity) and surface area. Useful modifying agents include solvents such as dichloromethane, trichloroacetic acid, acetonitrile and acids such as trifluoroacetic acid and hydrogen fluoride. Preferred fibril-modifying agent useful in formulating the matrices of this invention is a heated aqueous medium, preferably an acidic aqueous medium having a pH less than about pH 4.5, most preferably having a pH within the range of about pH 2-pH 4. Preferred heated acidic aqueous medium is 0.1% acetic acid which has a pH of about 3. Heating demineralized, delipidated, guanidine-extracted bone collagen in an aqueous medium at elevated temperatures (e.g., in the range of about 37° C.-65° C., preferably in the range of about 45° C.-60° C.) for approximately one hour generally is sufficient to achieve the desired surface morphology. Although the mechanism is not clear, it is hypothesized that the heat treatment alters the collagen fibrils, resulting in an increase in the particle surface area.

Demineralized guanidine-extracted xenogenic bovine bone comprises a mixture of additional materials that may be fractionated further using standard biomolecular purification techniques. For example, chromatographic separation of extract components followed by addition back to active matrix of the various extract fractions corresponding to the chromatogram peaks may be used to improve matrix properties by fractionating away inhibitors of bone or tissue-inductive activity.

The matrix may also be substantially depleted in residual heavy metals. Treated as disclosed herein, individual heavy metal concentrations in the matrix can be reduced to less than about 1 ppm.

One skilled in the art may create a biocompatible matrix of choice having a desired porosity or surface microtexture useful in the methods of this invention, or as a biodegradable sustained release implant. In addition, synthetically formulated matrices, prepared as disclosed herein, may be used.

Other Tissue-Specific Matrices

In addition to the naturally-derived bone matrices described above, useful matrices may also be formulated synthetically by adding together reagents that have been appropriately modified. One example of such a matrix is the porous, biocompatible, in vivo biodegradable synthetic matrix disclosed in WO91/18558, the disclosure of which is hereby incorporated by reference.

Briefly, the matrix comprises a porous crosslinked structural polymer of biocompatible, biodegradable collagen, most preferably tissue-specific collagen, and appropriate, tissue-specific glycosaminoglycans as tissue-specific cell attachment factors. Bone tissue-specific collagen (e.g., Type I collagen) derived from a number of sources may be suitable for use in these synthetic matrices, including soluble collagen, acid-soluble collagen, collagen soluble in neutral or basic aqueous solutions, as well as those collagens which are commercially available. In addition, Type II collagen, as found in cartilage, also may be used in combination with Type I collagen.

Glycosaminoglycans (GAGs) or mucopolysaccharides are polysaccharides made up of residues of hexoamines glycosidically bound and alternating in a more-or-less regular manner with either hexouronic acid or hexose moieties. GAGs are of animal origin and have a tissue specific distribution (see, e.g., Dodgson et al., in Carbohydrate Metabolism and its Disorders, Dickens et al., eds., Vol. 1, Academic Press (1968)). Reaction with the GAGs also provides collagen with another valuable property, i.e., inability to provoke an immune reaction (foreign body reaction) from an animal host.

Useful GAGs include those containing sulfate groups, such as hyaluronic acid, heparin, heparin sulfate, chondroitin 6-sulfate, chondroitin 4-sulfate, dermatan sulfate, and keratin sulfate. For osteogenic devices, chondroitin 6-sulfate currently is preferred. Other GAGs also may be suitable for forming the matrix described herein, and those skilled in the art will either know or be able to ascertain other suitable GAGs using no more than routine experimentation. For a more detailed description of mucopolysaccharides, see Aspinall, Polysaccharides, Pergamon Press, Oxford (1970).

Collagen can be reacted with a GAG in aqueous acidic solutions, preferably in diluted acetic acid solutions. By adding the GAG dropwise into the aqueous collagen dispersion, coprecipitates of tangled collagen fibrils coated with GAG results. This tangled mass of fibers then can be homogenized to form a homogeneous dispersion of fine fibers and then filtered and dried.

Insolubility of the collagen-GAG products can be raised to the desired degree by covalently cross-linking these materials, which also serves to raise the resistance to resorption of these materials. In general, any covalent G60 cross-linking method suitable for cross-linking collagen also is suitable for cross-linking these composite materials, although cross-linking by a dehydrothermal process is preferred.

When dry, the cross-linked particles are essentially spherical with diameters of about 500 μm. Scanning electron microscopy shows pores of about 20 μm on the surface and 40 μm on the interior. The interior is made up of both fibrous and sheet-like structures, providing surfaces for cell attachment. The voids interconnect, providing access to the cells throughout the interior of the particle. The material appears to be roughly 99.5% void volume, making the material very efficient in terms of the potential cell mass that can be grown per gram of microcarrier.

Another useful synthetic matrix is one formulated from biocompatible, in vivo biodegradable synthetic polymers, such as those composed of glycolic acid, lactic acid and/or butyric acid, including copolymers and derivatives thereof. These polymers are well described in the art and are available commercially. For example, polymers composed of polylactic acid (e.g., MW 100 ka), 80% polylactide/20% glycoside or poly 3-hydroxybutyric acid (e.g., MW 30 ka) all may be purchased from PolySciences, Inc. The polymer compositions generally are obtained in particulate form and the morphogenic devices preferably fabricated under nonaqueous conditions (e.g., in an ethanol-trifluoroacetic acid solution, EtOH/TFA) to avoid hydrolysis of the polymers. In addition, one can alter the morphology of the particulate polymer compositions, for example to increase porosity, using any of a number of particular solvent treatments known in the art.

### Example 1

### Cell Culture and Cell Morphology Study

Three human tumor cells lines were used as models: osteosarcoma cell lines SaOS-2 and MG-63 and lung carcinoma cell line A549. Cell lines were obtained from American Tissue Culture Collection (ATCC) and were grown and sustained in the appropriate media using standard techniques. Morphology of the cultured cells was monitored with an Olympus CK2 inverted microscope equipped with a CCD camera. Images were captured using phase contrast with 100× magnification.

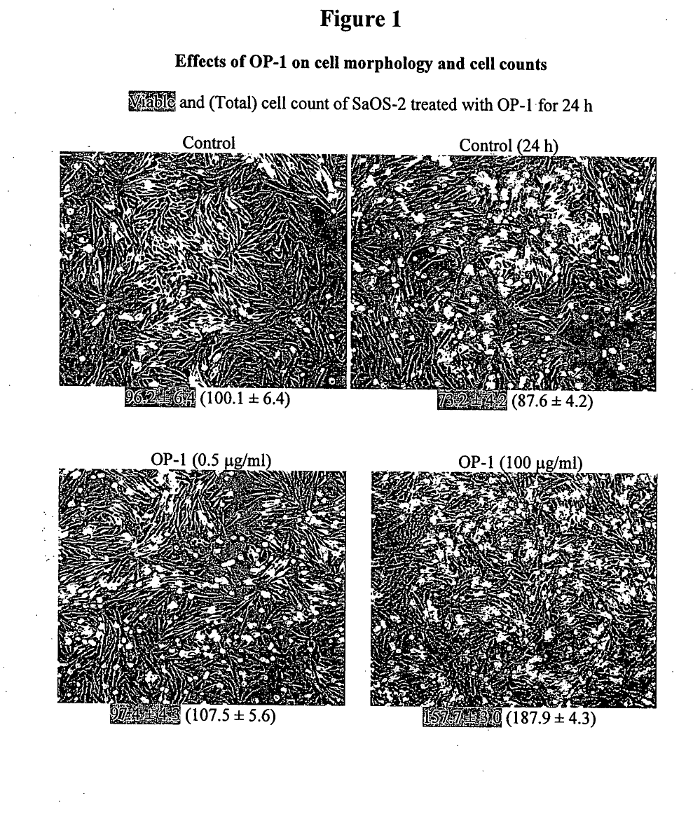

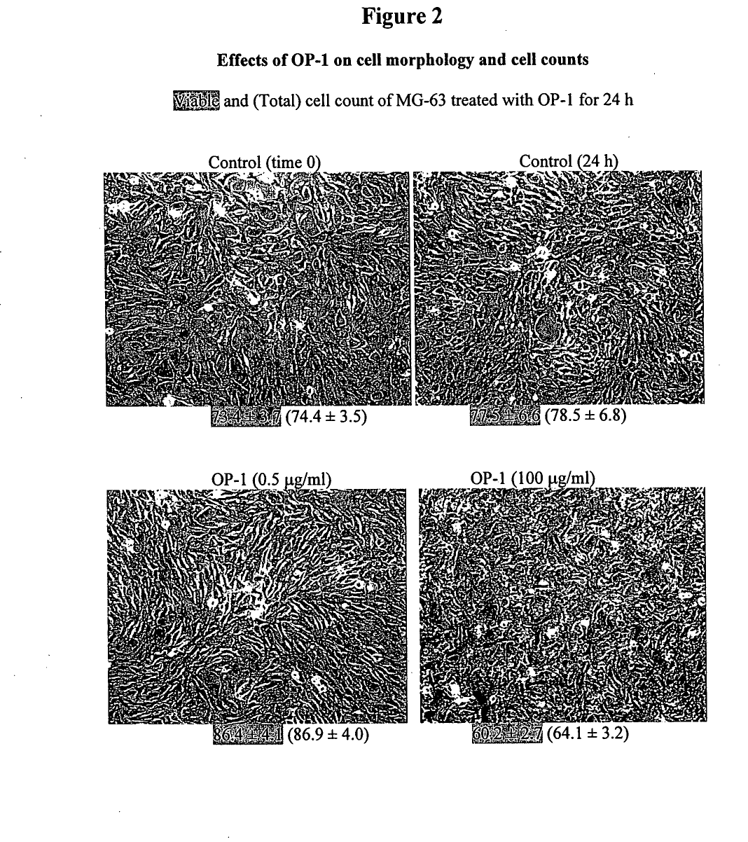

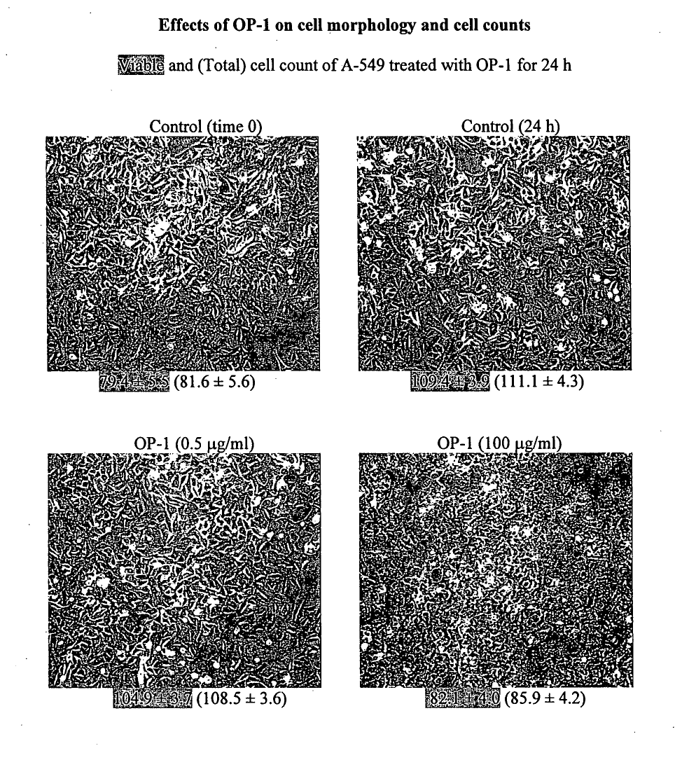

To examine the effects of OP-1 on cell morphology and cell counts, cells were treated with different concentrations of OP-1 (0.5 μg/ml and 100 μg/ml) for 24 h. The viable and total cell counts following OP-1 treatment were measured using the trypan blue exclusion assay. Treatment of SaOS-2 cells with OP-1 increased both viable and total cell counts in a dose-dependent manner as compared with untreated control cells (see FIG. 1). OP-1 treatment of MG-63 cells did not increase viable and total cell counts significantly. Treatment with an OP-1 concentration of 100 μg/ml inhibited cell counts (see FIG. 2). Similarly, OP-1 treatment of A549 cells at a concentration of 0.5 μg/ml did not affect viable and total cell counts as compared with untreated control cells, but at the higher concentration of 100 μg/ml, OP-1 significantly reduced cell counts (see FIG. 3).

### Example 2

### BMP Receptor mRNA Expression

Total RNA was isolated using TRI reagent (Molecular Research Center, Inc., Cincinnati, Ohio) following the manufacturer's recommendation. The cDNA probes for ActR-I, BMPR-IA, BMPR-IB, and BMPR-II were obtained by digestion of the corresponding plasmids with the appropriate restriction endonucleases as reported previously (Yeh et al., J Cell Physiol 185:87-97 (2000). Specifically, the 580-bp ActR-I insert was obtained by digestion of the parent plasmid containing the ActR-I insert with EcoRI/Aval. The 530-bp BMPR-IA insert was obtained by digestion with HindIII/PvuII. The 660-bp BMPR-IB insert was obtained by digestion with HpaI/SacI. The 800-bp BMPR-II insert was obtained by PstI digestion of hBMPR-II cloned in pCMV5. The resultant cDNA fragments were purified by agarose gel electrophoresis and were labeled with [α-³²P]dATP using the Strip-EZ DNA labeling system (Ambion Co, Austin, Tex.). The labeled cDNA probes were purified through a Midi-SELECT G-25 spin column (IBI, New Haven, Conn.) to remove the un-incorporated nucleotides. The 18S rRNA was probed with a ³²P-labeled, 18S-specific oligonucleotide with the following sequence: 5′-GCCGTGCGTACTTAGACATGCATG-3′ (SEQ ID NO: 10).

Northern analyses were conducted to probe for the presence of mRNA for ActR-I, BMPR-IA, BMPR-IB and BMPR-II on osteosarcoma cell lines SaOS-2 and MG-63, lung carcinoma cell line A549, and prostate carcinoma cell line PC-3 as described previously using 20 μg of total RNAs. Briefly, 20 μg of total RNAs were denatured and analyzed on 2.2M formaldehyde/1% GTG agarose gels. RNA standards (0.24-9.5 kb) from Life technologies (Grand Island, N.Y.) were used as size markers. After electrophoresis, the fractionated RNAs were transferred onto a “Nytran Plus” membrane using a Turboblot apparatus (Schleicher & Schuell, Inc., Keene, N.H.). After cutting the lane containing the standards from the blot, the RNAs were covalently linked to the membrane using the UV Crosslinker (Stratagene, La Jolla, Calif.). The membranes were incubated overnight at 42° C. with a particular cDNA probe containing one of the OP-1 receptor sequences. The blots were washed and exposed to a screen for the PhosphorImager (Molecular Dynamics, Sunnyvale, Calif.) to detect the hybridized signals. The same blots were washed with Strip-EZ Degradation Buffer (Ambion, Austin, Tex.) to remove the probe followed by sequential probing with the remaining receptor cDNA probes. Blots were also probed with an 18S rRNA oligonucleotide probe to correct for loading variations. The intensity of the hybridization signal was analyzed and quantified using the ImageQuant software and corrected for the intensity of the rRNA. Each experiment was conducted four times.

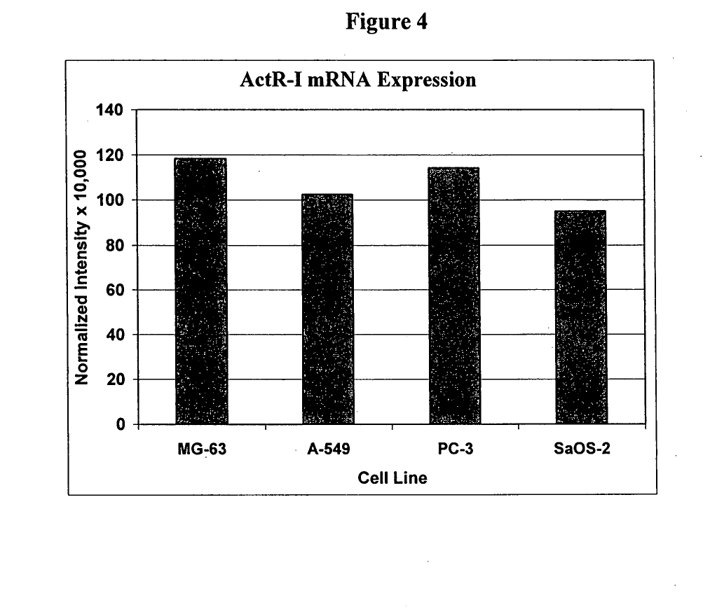

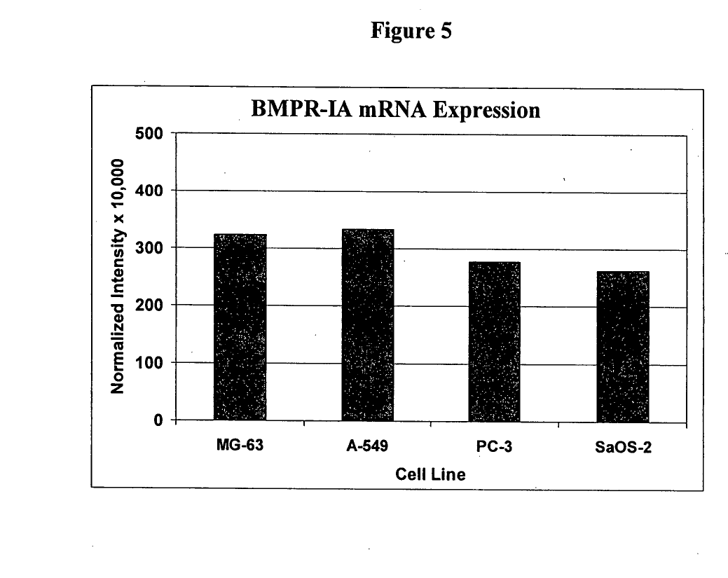

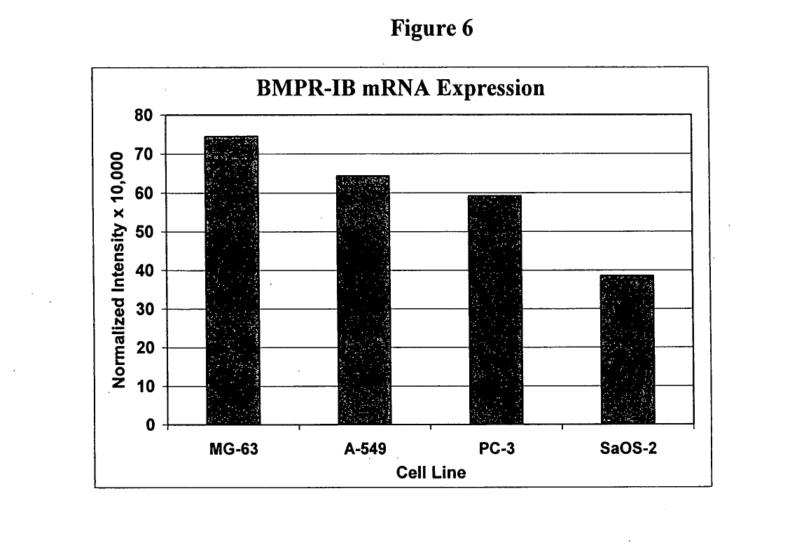

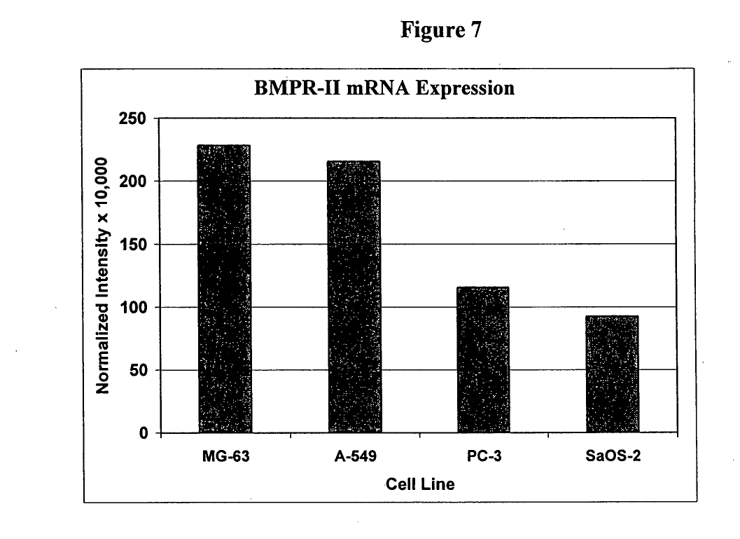

Northern blot analysis revealed that all four cell lines expressed mRNA coding for ActR-IA, BMPR-IA, BMPR-IB, and BMPR-II, though to varying degrees (see FIGS. 4-7).

### Example 3

### Effects of OP-1 on Cell Proliferation In Vitro

To examine the effects of OP-1 on cell proliferation, cells were subcultured at a cell density of 2×10⁴/ml in a 48-well plate and grown in the appropriate medium with serum until mid-log phase. The specific day at which the culture reached mid-log (the doubling time) varied according to the individual cell line. Cells were then treated with various concentrations of OP-1 (0, 0.1, 0.5, 1.0, 5.0, 10, 50, and 100 μg/ml; Stryker Biotech, Hopkinton, Mass.) in serum free-medium containing 0.1% BSA for 18 h. Cells were pulsed with [³H] thymidine for 6 h after treatment (with the exception of the SaOS-2 cell line in which cell proliferation was evaluated by a tetrazolium calorimetric assay; see discussion below). The extent of incorporation of [³H]thymidine (5 μCi/ml) into DNA and the number of cells were determined as previously described (Yeh et al., Endocrinology 138:4181-4190 (1997)). After removal of the medium containing the unincorporated thymidine, cells were rinsed with cold 1×PBS. The radiolabeled DNA was precipitated by cold 10% TCA for 15 min, solubilized in 0.1N NaOH at 37° C. for 10 min, and neutralized with 0.1N HCl. The amount of radioactivity was determined by scintillation spectrometry in the presence of Econo-Safe cocktail (5 ml). The rate of cellular proliferation of the OP-1-treated samples was optionally defined as a percentage of the solvent-treated control.

Cell proliferation was also evaluated by a tetrazolium colorimetric assay (CellTiter96AQ Cell Proliferation Assay, Promega, Madison, Wis.) following the manufacturer's instruction. Briefly, SaOS-2 cells were cultured in 96-well plates and treated in the presence of different concentrations of OP-1 for 24 h beginning on the pre-determined day that depended on the doubling time of the specific cell line. Media were removed from the cultures and rinsed with sterile PBS followed by the addition to each well of 100 μl of medium containing 1% BSA plus 20 μl of the 96AQ reagent. After 4 h, the color developed was measured at 490 nm using a MRX microplate reader (Dynex Technologies, Chantilly, Va.).

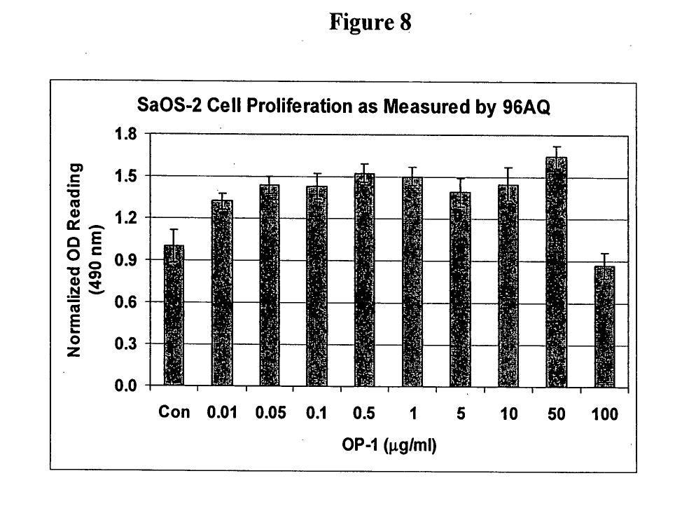

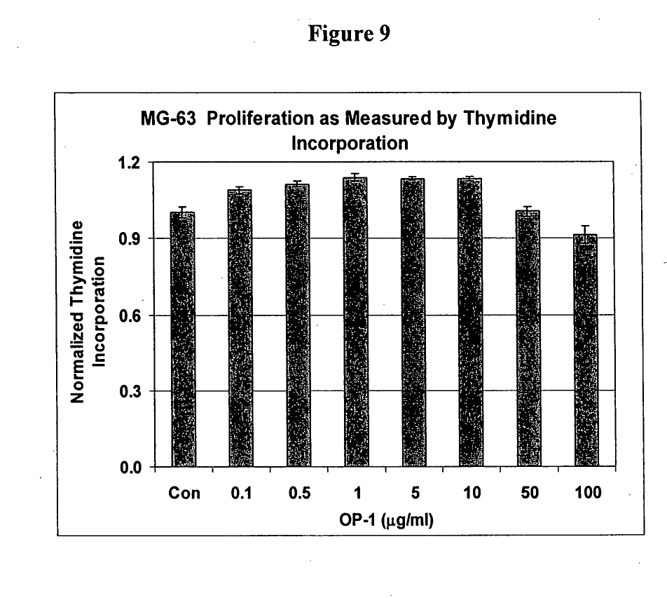

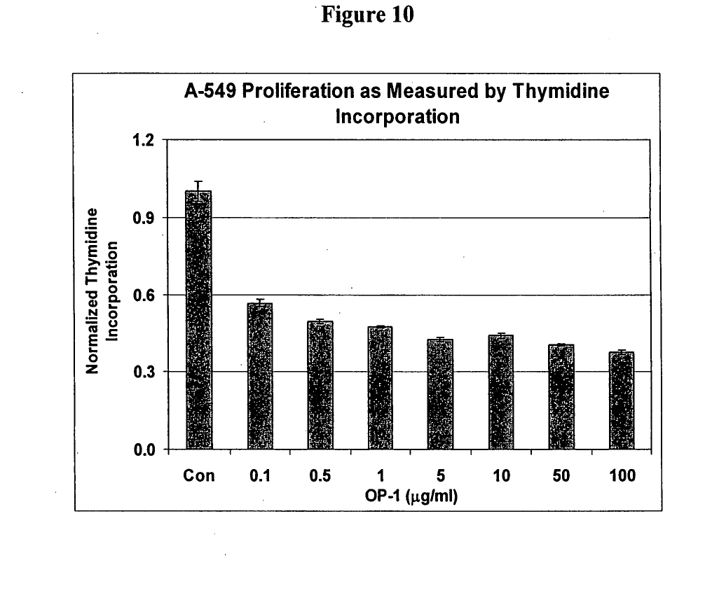

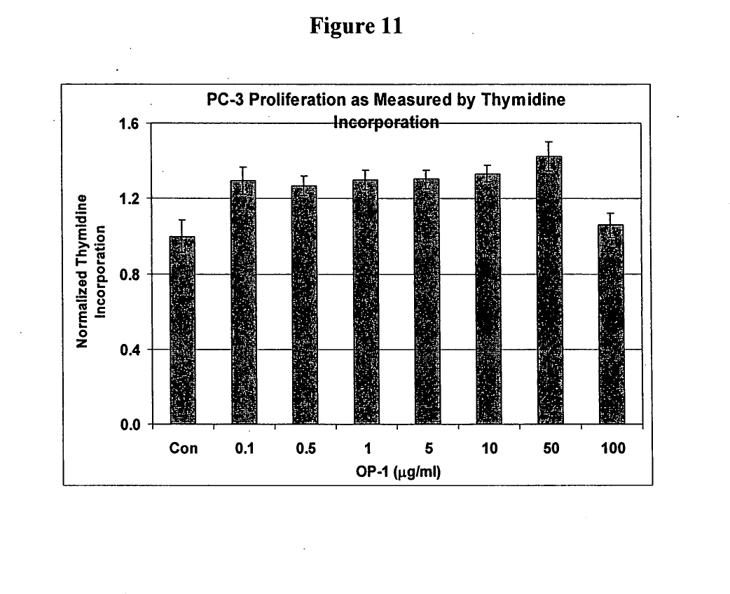

Treatment of SaOS-2 cells with OP-1 increased the number of cells in a dose-dependent manner (see FIG. 8). OP-1 treatment of MG-63 cells did not affect thymidine incorporation or cell number significantly; in fact, high OP-1 concentrations inhibited cell proliferation significantly (see FIG. 9). OP-1 treatment of A549 cells reduced thymidine incorporation and number of cells (see FIG. 10). In PC-3 cells, OP-1 stimulated thymidine incorporation by a maximum of 40% above the control, but at high protein concentrations, OP-1 inhibited thymidine incorporation (see FIG. 11). After treatment with high concentrations of OP-1 for 24 h, all four cell lines showed abnormal appearance, exhibiting cytopathological changes, indicating cell death compared to control cultures.

### Example 4

### Alkaline Phosphatase Activity Assay

Briefly, cells were grown and treated in 48-well plates with various concentrations of OP-1 (0, 0.1, 0.5, 1.0, 5.0, 10, 50, and 100 μg/ml; Stryker Biotech, Hopkinton, Mass.) for 48 h. After the 48 h treatment period, cells were rinsed with PBS and lysed by sonication in 100 μl/well 0.05% Triton X-100 in PBS, (250 μl/well for SaOS-2) for 5 min at room temperature. Total cellular AP activity was measured with p-nitrophenyl phosphate as a substrate in 2-amino-2-methyl-1-propanol buffer, pH 10.3 at 37° C. using a commercial assay kit (Sigma Chemical Co.). Reactions were terminated by the addition of 0.5N NaOH. Absorbance of the reaction mixture was measured at 405 nm using a MRX microplate reader (Dynex Technologies, Chantilly, Va.). Protein was measured according to the method of Bradford using BSA as a standard. AP activity was expressed as nanomoles of p-nitrophenol liberated per μg of total cellular protein. The total AP activity of the OP-1-treated samples was normalized to the solvent-treated control as 1.

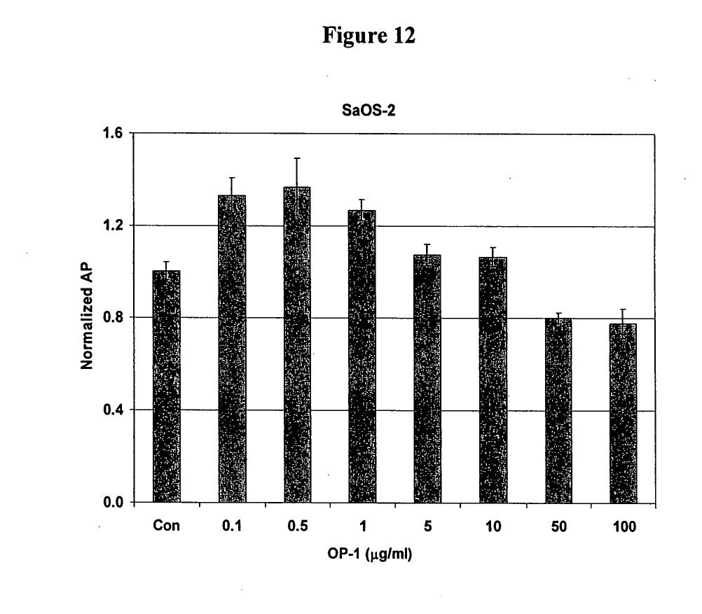

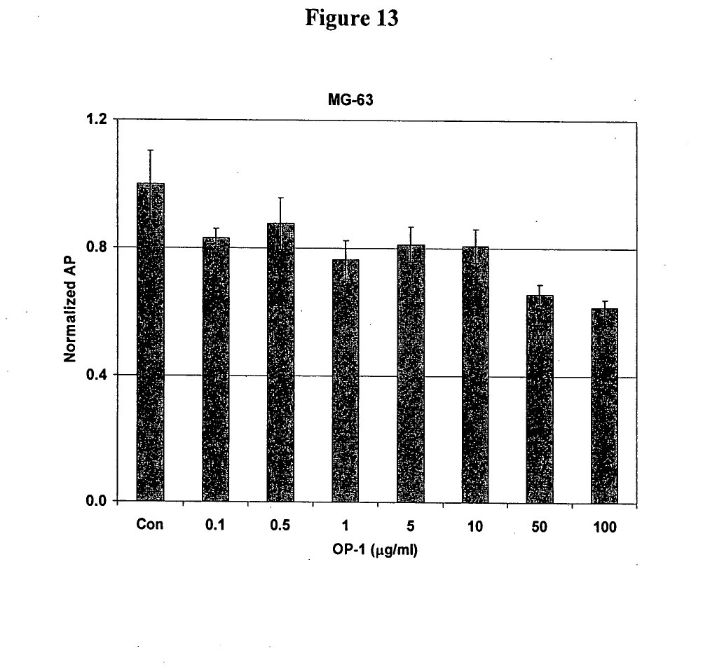

Treatment of both osteosarcoma cell lines SaOS-2 and MG-63 down-regulated alkaline phosphatase activity, a biochemical marker of osteoblastic cell differentiation (see FIGS. 12 and 13).

### Example 5

### Effects of OP-1 on Tumor Cell Growth and Differentiation In Vivo in a Nude Mouse Xenograft Model

To examine the effect of OP-1 on tumor cell growth and differentiation in vivo, tumor cell lines were grown to mid-log in complete media. Cells were then treated with 100 μg/ml of OP-1 for 24 h. After the 24h treatment period, cells were collected, washed, resuspended in MEM, and injected subcutaneously (10⁶ cells in 0.1 ml HBSS) into the flank of young adult male BALB/C athymic nude mice (treated group). The control group for each tumor cell line tested received a single subcutaneous injection of tumor cells (10⁶ cells in 0.1 ml HBSS) with exposure to OP-1. Mice were monitored daily for general health and the development/progression of tumors via in-life measurement of tumor mass for 49 days after which they were necropised. The size of the tumor mass as a function of time is provided for the different tumor cell lines tested. Masses, when present, were excised, measured, and fixed in 10% neutral buffered formalin. Sections of liver, lung, and skin at injection site were also collected and fixed. Fixed tissue samples were embedded in paraffin, sectioned at 5μ, stained with hematoxylin and eosin (H&E), and examined microscopically.

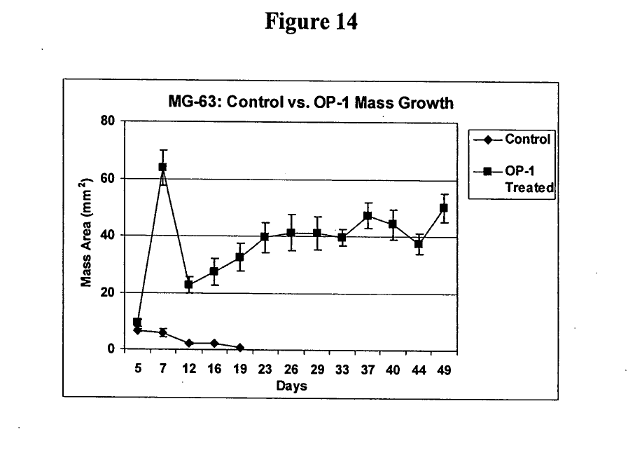

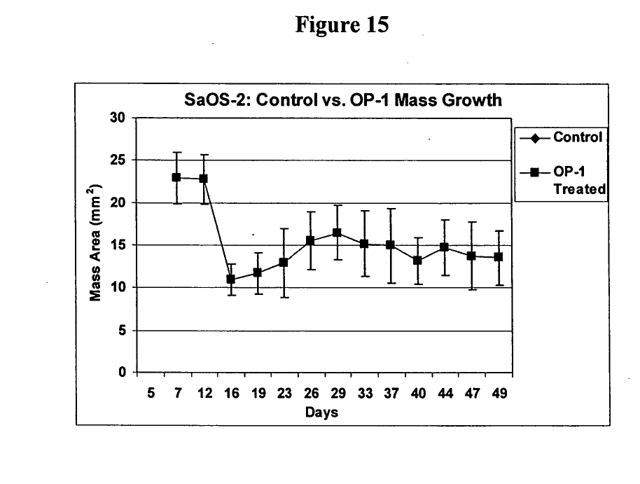

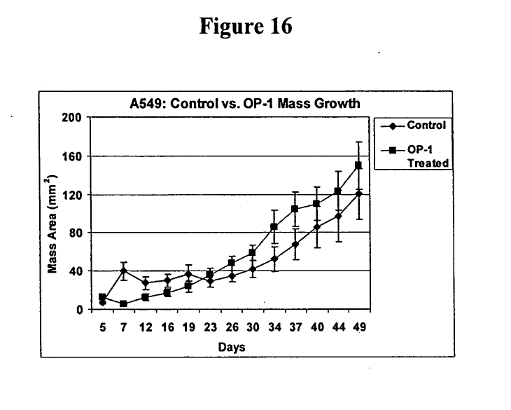

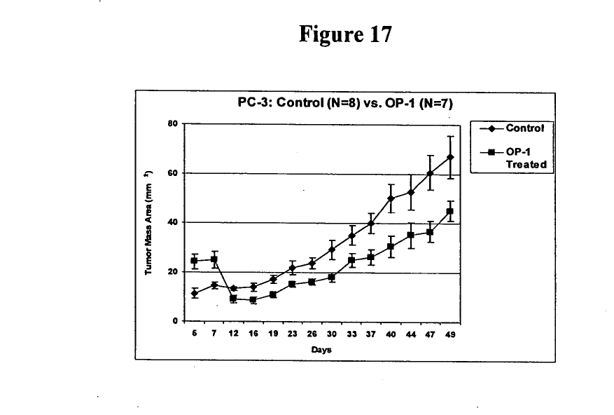

No masses were detected at the injection site in mice injected with either control MG-63 (see FIG. 14) or SaOS-2 (see FIG. 15) cell lines. In mice injected with OP-1-treated MG-63 osteosarcoma cells, tissue masses were detected at the site of injection and increased in size as a function of time (see FIG. 14). In the OP-1-treated SaOS-2 osteosarcoma group, mice exhibited masses at the site of injection; however, the mass size decreased as a function of time (see FIG. 15). In contrast, masses were detected at the injection site in mice injected with either control A549 (see FIG. 16) or PC-3 (see FIG. 17) cell lines. Upon injection with OP-1-treated A549 or PC-3 cells, tissue masses increased in size as a function of time (see FIGS. 16 and 17, respectively). In 8 of 8 mice injected with OP-1 -treated MG-63 osteosarcoma cells, tissue masses were detected at the site of injection and increased in size as a function of time.

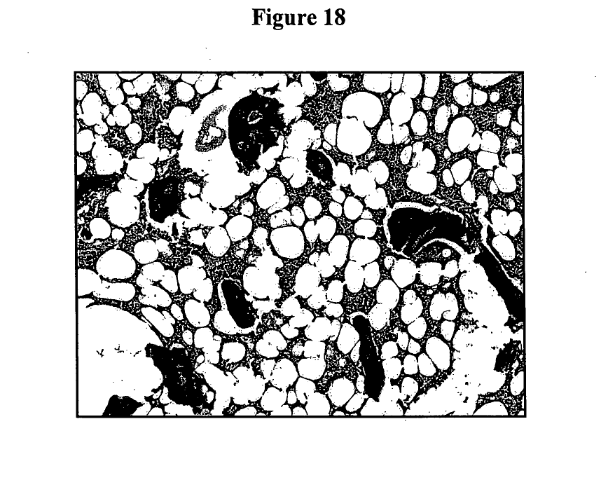

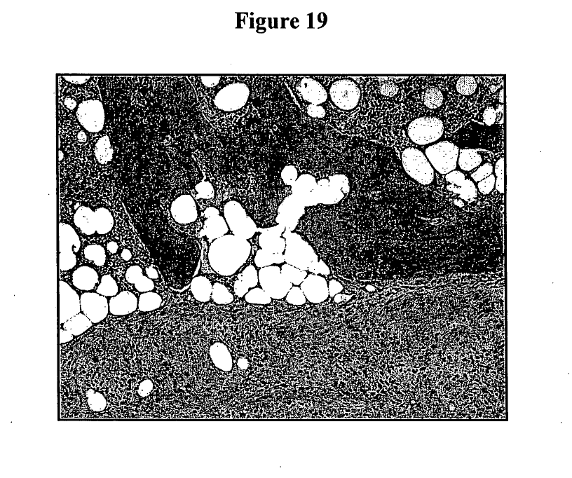

Microscopically, these masses consisted of mature bone formation with or without the presence of active bone marrow, as depicted in FIG. 18 which shows the histologic appearance of a subcutaneous bone nodule containing bone marrow. In the OP-1-treated MG-63 osteosarcoma cell group, one animal displayed several tumor cell foci within the bone nodule (see FIG. 19).

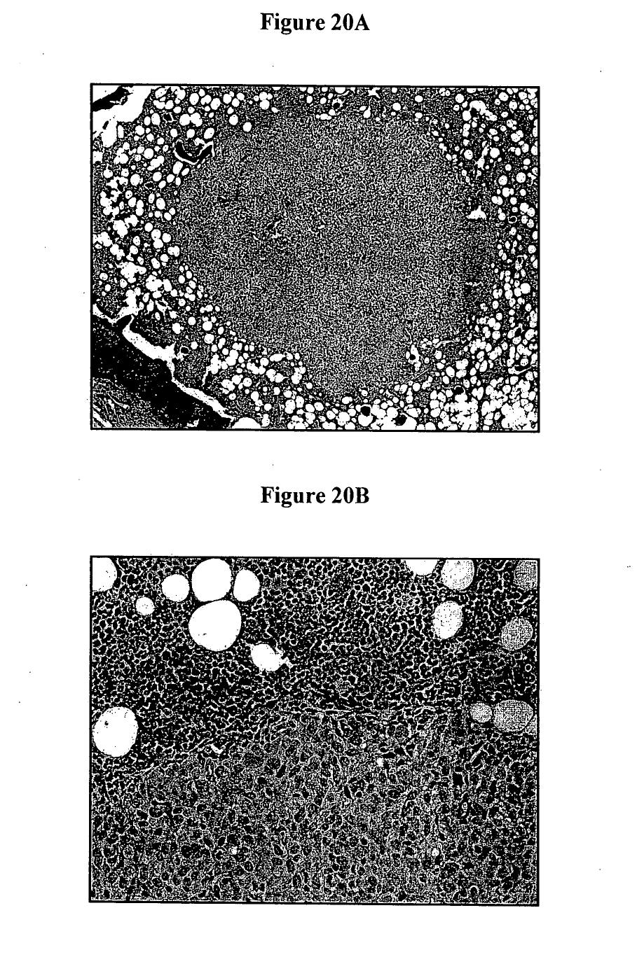

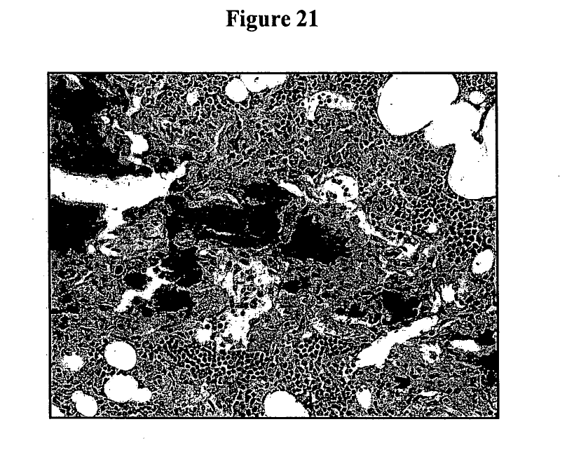

In the OP-1-treated SaOS-2 group, 5 of 8 mice exhibited masses at site of injection. Histologically, these masses consisted of well differentiated ectopic bone and bone marrow. In some cases, the bone surrounded a small, well-contained focus of tumor cells (see FIGS. 20A and 20B). In other cases, the tissue masses consisted of a nodule of ectopic bone that contained a small focus of tumor cells that appeared to be regressing (see FIG. 21).

In a separate series of experiments, the effects of co-administration of OP-1 and PC-3 cells on the development and progression of tumor formation as compared to either PC-3 cells alone or OP-1 alone were examined. Microscopic examination of the masses from mice injected with OP-1 alone exhibited ectopic bone containing bone marrow at the site of injection in all mice (8 of 8). Mice injected with PC-3 cells only showed carcinomas at the site of injection in 8 of 8 mice. Mice injected with a mixture of OP-1 and PC-3 cells showed subcutaneous masses in 8 of 8 mice. Microscopic examination of these masses showed ectopic bone formation with some tumor cell foci in or adjacent to the bone nodule. Mice injected with control PC-3 cells exhibited tumor at the site of injection. In summary, the present study using four diverse human tumor cell lines in a nude mouse xenograft model shows that OP-1 does not stimulate tumor cell growth and metastasis. Moreover, at high concentrations, OP-1 suppresses tumor cell differentiation and stimulates ectopic bone formation.

What is claimed is:

1. A method of treating bone cancer in a mammal comprising the step of administering to the mammal a pharmaceutically effective amount of a bone morphogenic protein or a nucleic acid encoding the bone morphogenic protein.

2. A method of inducing differentiation of bone tumor cells in a mammal comprising the step of administering to the mammal a pharmaceutically effective amount of a bone morphogenic protein or a nucleic acid encoding the bone morphogenic protein.

3. A method of inhibiting bone tumor growth in a mammal comprising the step of administering to the mammal a pharmaceutically effective amount of a bone morphogenic protein or a nucleic acid encoding the bone morphogenic protein.

4. A method of inducing bone tumor regression in a mammal comprising the step of administering to the mammal a pharmaceutically effective amount of a bone morphogenic protein or a nucleic acid encoding the bone morphogenic protein.

5. The method according to any one of claims 1-4, wherein the bone tumor is a sarcoma or a carcinoma.

6. The method according to claim 5, wherein the sarcoma is an osteosarcoma.

7. The method according to any one of claims 1-4, wherein the bone morphogenic protein is selected from the group consisting of OP-1, OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17, BMP-18, DPP, Vg1, Vgr, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, NODAL, UNIVIN, SCREW, ADMP, NEURAL, and fragments thereof.

8. The method according to claim 7, wherein the bone morphogenic protein is selected from the group consisting of OP-1, BMP-5, BMP-6, GDF-5, GDF-6, GDF-7, CDMP-1, CDMP-2 and CDMP-3.

9. The method according to claim 8, wherein the bone morphogenic protein is selected from the group consisting of OP-1, BMP-5 and BMP-6.

10. The method according to claim 9 wherein the bone morphogenic protein is OP-1.

11. The method according to claims 1-4, wherein the nucleic acid is a viral vector comprising a gene that encodes the bone morphogenic protein, and wherein the viral vector is selected from the group consisting of an adenoviral vector, a lentiviral vector, a baculoviral vector, an Epstein Barr viral vector, a papovaviral vector, a vaccinia viral vector and a herpes simplex viral vector.

12. The method according to claim 11, wherein the viral vector is selected from the group consisting of an adenoviral vector, a baculoviral vector and a lentiviral vector.

13. The method according to claim 12, wherein the viral vector is an adenoviral vector.

14. The method according to any one of claims 1-4, wherein the bone morphogenic protein or nucleic acid encoding the bone morphogenic protein is formulated as a gel, an aqueous solution, a paste or a putty.

15. The method according to claim 14, wherein the formulation is a sustained release formulation.

16. The method according to claim 14, wherein the bone morphogenic protein or nucleic acid encoding the bone morphogenic protein is formulated for local administration.


SEQUENCE LISTING (18 sequences)

SEQ ID NO: 1 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETERISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 2 - protein, 96 aa, Artificial Sequence
  Description of Artificial Sequence Synthetic polypeptide
LYVDFSDVGWDDWIVAPPGYQAFYCHGECPFPLADHFNSTNHAVVQTLVNSVNSKIPKACCVPTELSAISMLYLDENEKVVLKYNQEMVVEGCGCR

SEQ ID NO: 3 - protein, 96 aa, Artificial Sequence
  Description of Artificial Sequence Synthetic polypeptide
LYVDFSDVGWNDWIVAPPGYHAFYCHGECPFPLADHLNSTNHAVVQTLVNSVNSKIPKACCVPTELSAISMLYLDENEKVVLKYNQEMVVEGCGCR

SEQ ID NO: 4 - protein, 102 aa, Artificial Sequence
  Description of Artificial Sequence Synthetic polypeptide
  MOD_RES (2)..(3): Lys or Arg
  MOD_RES (11)..(11): Arg or Gln
  MOD_RES (16)..(16): Gln or Leu
  MOD_RES (19)..(19): Ile or Val
  MOD_RES (23)..(23): Glu or Gln
  MOD_RES (26)..(26): Ala or Ser
  MOD_RES (35)..(35): Ala or Ser
  MOD_RES (39)..(39): Asn or Asp
  MOD_RES (41)..(41): Tyr or Cys
  MOD_RES (50)..(50): Val or Leu
  MOD_RES (52)..(52): Ser or Thr
  MOD_RES (56)..(56): Phe or Leu
  MOD_RES (57)..(57): Ile or Met
  MOD_RES (58)..(58): Asn or Lys
  MOD_RES (60)..(60): Glu, Asp or Asn
  MOD_RES (61)..(61): Thr, Ala or Val
  MOD_RES (65)..(65): Pro or Ala
  MOD_RES (71)..(71): Gln or Lys
  MOD_RES (73)..(73): Asn or Ser
  MOD_RES (75)..(75): Ile or Thr
  MOD_RES (80)..(80): Phe or Tyr
  MOD_RES (82)..(82): Asp or Ser
  MOD_RES (84)..(84): Ser or Asn
  MOD_RES (89)..(89): Lys or Arg
  MOD_RES (91)..(91): Tyr or His
  MOD_RES (97)..(97): Arg or Lys
CXXHELYVSFXDLGWXDWXIAPXGYXAYYCEGECXFPLXSXMNATNHAIXQXLVHXXXPXXVPKXCCAPTXLXAXSVLYXDXSXNVILXKXRNMVVXACGCH

SEQ ID NO: 5 - protein, 97 aa, Artificial Sequence
  Description of Artificial Sequence Synthetic polypeptide
  MOD_RES (2)..(2): Tyr or Lys
  MOD_RES (3)..(3): Val or Ile
  MOD_RES (4)..(4): Ser, Asp or Glu
  MOD_RES (6)..(6): Arg, Gln, Ser, Lys or Ala
  MOD_RES (7)..(7): Asp or Glu
  MOD_RES (8)..(8): Leu, Val or Ile
  MOD_RES (11)..(11): Gln, Leu, Asp, His, Asn or Ser
  MOD_RES (12)..(12): Asp, Arg, Asn or Glu
  MOD_RES (13)..(13): Trp or Ser
  MOD_RES (14)..(15): Ile or Val
  MOD_RES (16)..(16): Ala or Ser
  MOD_RES (18)..(18): Glu, Gln, Leu, Lys, Pro or Arg
  MOD_RES (19)..(19): Gly or Ser
  MOD_RES (20)..(20): Tyr or Phe
  MOD_RES (21)..(21): Ala, Ser, Asp, Met, His, Gln, Leu or Gly
  MOD_RES (23)..(23): Tyr, Asn or Phe
  MOD_RES (26)..(26): Glu, His, Tyr, Asp, Gln, Ala or Ser
  MOD_RES (28)..(28): Glu, Lys, Asp, Gln or Ala
  MOD_RES (30)..(30): Ala, Ser, Pro, Gln, Ile or Asn
  MOD_RES (31)..(31): Phe, Leu or Tyr
  MOD_RES (33)..(33): Leu, Val or Met
  MOD_RES (34)..(34): Asn, Asp, Ala, Thr or Pro
  MOD_RES (35)..(35): Ser, Asp, Glu, Leu, Ala or Lys
  MOD_RES (36)..(36): Tyr, Cys, His, Ser or Ile
  MOD_RES (37)..(37): Met, Phe, Gly or Leu
  MOD_RES (38)..(38): Asn, Ser or Lys
  MOD_RES (39)..(39): Ala, Ser, Gly or Pro
  MOD_RES (40)..(40): Thr, Leu or Ser
  MOD_RES (44)..(44): Ile, Val or Thr
  MOD_RES (45)..(45): Val, Leu, Met or Ile
  MOD_RES (46)..(46): Gln or Arg
  MOD_RES (47)..(47): Thr, Ala or Ser
  MOD_RES (48)..(48): Leu or Ile
  MOD_RES (49)..(49): Val or Met
  MOD_RES (50)..(50): His, Asn or Arg
  MOD_RES (51)..(51): Phe, Leu, Asn, Ser, Ala or Val
  MOD_RES (52)..(52): Ile, Met, Asn, Ala, Val, Gly or Leu
  MOD_RES (53)..(53): Asn, Lys, Ala, Glu, Gly or Phe
  MOD_RES (54)..(54): Pro, Ser or Val
  MOD_RES (55)..(55): Glu, Asp, Asn, Gly, Val, Pro or Lys
  MOD_RES (56)..(56): Thr, Ala, Val, Lys, Asp, Tyr, Ser, Gly, Ile or His
  MOD_RES (57)..(57): Val, Ala or Ile
  MOD_RES (58)..(58): Pro or Asp
  MOD_RES (59)..(59): Lys, Leu or Glu
  MOD_RES (60)..(60): Pro, Val or Ala
  MOD_RES (63)..(63): Ala or Val
  MOD_RES (65)..(65): Thr, Ala or Glu
  MOD_RES (66)..(66): Gln, Lys, Arg or Glu
  MOD_RES (67)..(67): Leu, Met or Val
  MOD_RES (68)..(68): Asn, Ser, Asp or Gly
  MOD_RES (69)..(69): Ala, Pro or Ser
  MOD_RES (70)..(70): Ile, Thr, Val or Leu
  MOD_RES (71)..(71): Ser, Ala or Pro
  MOD_RES (72)..(72): Val, Leu, Met or Ile
  MOD_RES (74)..(74): Tyr or Phe
  MOD_RES (75)..(75): Phe, Tyr, Leu or His
  MOD_RES (76)..(76): Asp, Asn or Leu
  MOD_RES (77)..(77): Asp, Glu, Asn, Arg or Ser
  MOD_RES (78)..(78): Ser, Gln, Asn, Tyr or Asp
  MOD_RES (79)..(79): Ser, Asn, Asp, Glu or Lys
  MOD_RES (80)..(80): Asn, Thr or Lys
  MOD_RES (82)..(82): Ile, Val or Asn
  MOD_RES (84)..(84): Lys or Arg
  MOD_RES (85)..(85): Lys, Asn, Gln, His, Arg or Val
  MOD_RES (86)..(86): Tyr, Glu or His
  MOD_RES (87)..(87): Arg, Gln, Glu or Pro
  MOD_RES (88)..(88): Asn, Glu, Trp or Asp
  MOD_RES (90)..(90): Val, Thr, Ala or Ile
  MOD_RES (92)..(92): Arg, Lys, Val, Asp, Gln or Glu
  MOD_RES (93)..(93): Ala, Gly, Glu or Ser
  MOD_RES (95)..(95): Gly or Ala
  MOD_RES (97)..(97): His or Arg
LXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 6 - protein, 102 aa, Artificial Sequence
  Description of Artificial Sequence Synthetic polypeptide
  MOD_RES (2)..(5): Variable amino acid
  MOD_RES (7)..(7): Tyr or Lys
  MOD_RES (8)..(8): Val or Ile
  MOD_RES (9)..(9): Ser, Asp or Glu
  MOD_RES (11)..(11): Arg, Gln, Ser, Lys or Ala
  MOD_RES (12)..(12): Asp or Glu
  MOD_RES (13)..(13): Leu, Val or Ile
  MOD_RES (16)..(16): Gln, Leu, Asp, His, Asn or Ser
  MOD_RES (17)..(17): Asp, Arg, Asn or Glu
  MOD_RES (18)..(18): Trp or Ser
  MOD_RES (19)..(20): Ile or Val
  MOD_RES (21)..(21): Ala or Ser
  MOD_RES (23)..(23): Glu, Gln, Leu, Lys, Pro or Arg
  MOD_RES (24)..(24): Gly or Ser
  MOD_RES (25)..(25): Tyr or Phe
  MOD_RES (26)..(26): Ala, Ser, Asp, Met, His, Gln, Leu or Gly
  MOD_RES (28)..(28): Tyr, Asn or Phe
  MOD_RES (31)..(31): Glu, His, Tyr, Asp, Gln, Ala or Ser
  MOD_RES (33)..(33): Glu, Lys, Asp, Gln or Ala
  MOD_RES (35)..(35): Ala, Ser, Pro, Gln, Ile or Asn
  MOD_RES (36)..(36): Phe, Leu or Tyr
  MOD_RES (38)..(38): Leu, Val or Met
  MOD_RES (39)..(39): Asn, Asp, Ala, Thr or Pro
  MOD_RES (40)..(40): Ser, Asp, Glu, Leu, Ala or Lys
  MOD_RES (41)..(41): Tyr, Cys, His, Ser or Ile
  MOD_RES (42)..(42): Met, Phe, Gly or Leu
  MOD_RES (43)..(43): Asn, Ser or Lys
  MOD_RES (44)..(44): Ala, Ser, Gly or Pro
  MOD_RES (45)..(45): Thr, Leu or Ser
  MOD_RES (49)..(49): Ile, Val or Thr
  MOD_RES (50)..(50): Val, Leu, Met or Ile
  MOD_RES (51)..(51): Gln or Arg
  MOD_RES (52)..(52): Thr, Ala or Ser
  MOD_RES (53)..(53): Leu or Ile
  MOD_RES (54)..(54): Val or Met
  MOD_RES (55)..(55): His, Asn or Arg
  MOD_RES (56)..(56): Phe, Leu, Asn, Ser, Ala or Val
  MOD_RES (57)..(57): Ile, Met, Asn, Ala, Val, Gly or Leu
  MOD_RES (58)..(58): Asn, Lys, Ala, Glu, Gly or Phe
  MOD_RES (59)..(59): Pro, Ser or Val
  MOD_RES (60)..(60): Glu, Asp, Asn, Gly, Val, Pro or Lys
  MOD_RES (61)..(61): Thr, Ala, Val, Lys, Asp, Tyr, Ser, Gly, Ile or His
  MOD_RES (62)..(62): Val, Ala or Ile
  MOD_RES (63)..(63): Pro or Asp
  MOD_RES (64)..(64): Lys, Leu or Glu
  MOD_RES (65)..(65): Pro, Val or Ala
  MOD_RES (68)..(68): Ala or Val
  MOD_RES (70)..(70): Thr, Ala or Glu
  MOD_RES (71)..(71): Gln, Lys, Arg or Glu
  MOD_RES (72)..(72): Leu, Met or Val
  MOD_RES (73)..(73): Asn, Ser, Asp or Gly
  MOD_RES (74)..(74): Ala, Pro or Ser
  MOD_RES (75)..(75): Ile, Thr, Val or Leu
  MOD_RES (76)..(76): Ser, Ala or Pro
  MOD_RES (77)..(77): Val, Leu, Met or Ile
  MOD_RES (79)..(79): Tyr or Phe
  MOD_RES (80)..(80): Phe, Tyr, Leu or His
  MOD_RES (81)..(81): Asp, Asn or Leu
  MOD_RES (82)..(82): Asp, Glu, Asn, Arg or Ser
  MOD_RES (83)..(83): Ser, Gln, Asn, Tyr or Asp
  MOD_RES (84)..(84): Ser, Asn, Asp, Glu or Lys
  MOD_RES (85)..(85): Asn, Thr or Lys
  MOD_RES (87)..(87): Ile, Val or Asn
  MOD_RES (89)..(89): Lys or Arg
  MOD_RES (90)..(90): Lys, Asn, Gln, His, Arg or Val
  MOD_RES (91)..(91): Tyr, Glu or His
  MOD_RES (92)..(92): Arg, Gln, Glu or Pro
  MOD_RES (93)..(93): Asn, Glu, Trp or Asp
  MOD_RES (95)..(95): Val, Thr, Ala or Ile
  MOD_RES (97)..(97): Arg, Lys, Val, Asp, Gln or Glu
  MOD_RES (98)..(98): Ala, Gly, Glu or Ser
  MOD_RES (100)..(100): Gly or Ala
  MOD_RES (102)..(102): His or Arg
CXXXXLXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 7 - protein, 97 aa, Artificial Sequence
  Description of Artificial Sequence Synthetic polypeptide
  MOD_RES (1)..(1): Phe, Leu or Glu
  MOD_RES (2)..(2): Tyr, Phe, His, Arg, Thr, Lys, Gln, Val or Glu
  MOD_RES (3)..(3): Val, Ile, Leu or Asp
  MOD_RES (4)..(4): Ser, Asp, Glu, Asn or Phe
  MOD_RES (5)..(5): Phe or Glu
  MOD_RES (6)..(6): Arg, Gln, Lys, Ser, Glu, Ala or Asn
  MOD_RES (7)..(7): Asp, Glu, Leu, Ala or Gln
  MOD_RES (8)..(8): Leu, Val, Met, Ile or Phe
  MOD_RES (9)..(9): Gly, His or Lys
  MOD_RES (10)..(10): Trp or Met
  MOD_RES (11)..(11): Gln, Leu, His, Glu, Asn, Asp, Ser or Gly
  MOD_RES (12)..(12): Asp, Asn, Ser, Lys, Arg, Glu or His
  MOD_RES (13)..(13): Trp or Ser
  MOD_RES (14)..(15): Ile or Val
  MOD_RES (16)..(16): Ala, Ser, Tyr or Trp
  MOD_RES (18)..(18): Glu, Lys, Gln, Met, Pro, Leu, Arg, His or Lys
  MOD_RES (19)..(19): Gly, Glu, Asp, Lys, Ser, Gln, Arg or Phe
  MOD_RES (20)..(20): Tyr or Phe
  MOD_RES (21)..(21): Ala, Ser, Gly, Met, Gln, His, Glu, Asp, Leu, Asn, Lys or Tyr
  MOD_RES (22)..(22): Ala or Pro
  MOD_RES (23)..(23): Tyr, Phe, Asn, Ala or Arg
  MOD_RES (24)..(24): Tyr, His, Glu, Phe or Arg
  MOD_RES (26)..(26): Glu, Asp, Ala, Ser, Tyr, His, Lys, Arg, Gln or Gly
  MOD_RES (28)..(28): Glu, Asp, Leu, Val, Lys, Gly, Thr, Ala or Gln
  MOD_RES (30)..(30): Ala, Ser, Ile, Asn, Pro, Glu, Asp, Phe, Gln or Leu
  MOD_RES (31)..(31): Phe, Tyr, Leu, Asn, Gly or Arg
  MOD_RES (32)..(32): Pro, Ser, Ala or Val
  MOD_RES (33)..(33): Leu, Met, Glu, Phe or Val
  MOD_RES (34)..(34): Asn, Asp, Thr, Gly, Ala, Arg, Leu or Pro
  MOD_RES (35)..(35): Ser, Ala, Glu, Asp, Thr, Leu, Lys, Gln or His
  MOD_RES (36)..(36): Tyr, His, Cys, Ile, Arg, Asp, Asn, Lys, Ser, Glu or Gly
  MOD_RES (37)..(37): Met, Leu, Phe, Val, Gly or Tyr
  MOD_RES (38)..(38): Asn, Glu, Thr, Pro, Lys, His, Gly, Met, Val or Arg
  MOD_RES (39)..(39): Ala, Ser, Gly, Pro or Phe
  MOD_RES (40)..(40): Thr, Ser, Leu, Pro, His or Met
  MOD_RES (41)..(41): Asn, Lys, Val, Thr or Gln
  MOD_RES (42)..(42): His, Tyr or Lys
  MOD_RES (43)..(43): Ala, Thr, Leu or Tyr
  MOD_RES (44)..(44): Ile, Thr, Val, Phe, Tyr, Met or Pro
  MOD_RES (45)..(45): Val, Leu, Met, Ile or His
  MOD_RES (46)..(46): Gln, Arg or Thr
  MOD_RES (47)..(47): Thr, Ser, Ala, Asn or His
  MOD_RES (48)..(48): Leu, Asn or Ile
  MOD_RES (49)..(49): Val, Met, Leu, Pro or Ile
  MOD_RES (50)..(50): His, Asn, Arg, Lys, Tyr or Gln
  MOD_RES (51)..(51): Phe, Leu, Ser, Asn, Met, Ala, Arg, Glu, Gly or Gln
  MOD_RES (52)..(52): Ile, Met, Leu, Val, Lys, Gln, Ala or Tyr
  MOD_RES (53)..(53): Asn, Phe, Lys, Glu, Asp, Ala, Gln, Gly, Leu or Val
  MOD_RES (54)..(54): Pro, Asn, Ser, Val or Asp
  MOD_RES (55)..(55): Glu, Asp, Asn, Lys, Arg, Ser, Gly, Thr, Gln, Pro or His
  MOD_RES (56)..(56): Thr, His, Tyr, Ala, Ile, Lys, Asp, Ser, Gly or Arg
  MOD_RES (57)..(57): Val, Ile, Thr, Ala, Leu or Ser
  MOD_RES (58)..(58): Pro, Gly, Ser, Asp or Ala
  MOD_RES (59)..(59): Lys, Leu, Pro, Ala, Ser, Glu, Arg or Gly
  MOD_RES (60)..(60): Pro, Ala, Val, Thr or Ser
  MOD_RES (61)..(61): Cys, Val or Ser
  MOD_RES (63)..(63): Ala, Val or Thr
  MOD_RES (65)..(65): Thr, Ala, Glu, Val, Gly, Asp or Tyr
  MOD_RES (66)..(66): Gln, Lys, Glu, Arg or Val
  MOD_RES (67)..(67): Leu, Met, Thr or Tyr
  MOD_RES (68)..(68): Asn, Ser, Gly, Thr, Asp, Glu, Lys or Val
  MOD_RES (69)..(69): Ala, Pro, Gly or Ser
  MOD_RES (70)..(70): Ile, Thr, Leu or Val
  MOD_RES (71)..(71): Ser, Pro, Ala, Thr, Asn or Gly
  MOD_RES (72)..(72): Val, Ile, Leu or Met
  MOD_RES (74)..(74): Tyr, Phe, Arg, Thr, Tyr or Met
  MOD_RES (75)..(75): Phe, Tyr, His, Leu, Ile, Lys, Gln or Val
  MOD_RES (76)..(76): Asp, Leu, Asn or Glu
  MOD_RES (77)..(77): Asp, Ser, Arg, Asn, Glu, Ala, Lys, Gly or Pro
  MOD_RES (78)..(78): Ser, Asn, Asp, Tyr, Ala, Gly, Gln, Met, Glu, Asn or Lys
  MOD_RES (79)..(79): Ser, Asn, Glu, Asp, Val, Lys, Gly, Gln or Arg
  MOD_RES (80)..(80): Asn, Lys, Thr, Pro, Val, Ile, Arg, Ser or Gln
  MOD_RES (81)..(81): Val, Ile, Thr or Ala
  MOD_RES (82)..(82): Ile, Asn, Val, Leu, Tyr, Asp or Ala
  MOD_RES (83)..(83): Leu, Tyr, Lys or Ile
  MOD_RES (84)..(84): Lys, Arg, Asn, Tyr, Phe, Thr, Glu or Gly
  MOD_RES (85)..(85): Lys, Arg, His, Gln, Asn, Glu or Val
  MOD_RES (86)..(86): Tyr, His, Glu or Ile
  MOD_RES (87)..(87): Arg, Glu, Gln, Pro or Lys
  MOD_RES (88)..(88): Asn, Asp, Ala, Glu, Gly or Lys
  MOD_RES (89)..(89): Met or Ala
  MOD_RES (90)..(90): Val, Ile, Ala, Thr, Ser or Lys
  MOD_RES (91)..(91): Val or Ala
  MOD_RES (92)..(92): Arg, Lys, Gln, Asp, Glu, Val, Ala, Ser or Thr
  MOD_RES (93)..(93): Ala, Ser, Glu, Gly, Arg or Thr
  MOD_RES (95)..(95): Gly, Ala or Thr
  MOD_RES (97)..(97): His, Arg, Gly, Leu or Ser
XXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 8 - protein, 102 aa, Artificial Sequence
  Description of Artificial Sequence Synthetic polypeptide
  MOD_RES (2)..(5): Variable amino acid
  MOD_RES (6)..(6): Phe, Leu or Glu
  MOD_RES (7)..(7): Tyr, Phe, His, Arg, Thr, Lys, Gln, Val or Glu
  MOD_RES (8)..(8): Val, Ile, Leu or Asp
  MOD_RES (9)..(9): Ser, Asp, Glu, Asn or Phe
  MOD_RES (10)..(10): Phe or Glu
  MOD_RES (11)..(11): Arg, Gln, Lys, Ser, Glu, Ala or Asn
  MOD_RES (12)..(12): Asp, Glu, Leu, Ala or Gln
  MOD_RES (13)..(13): Leu, Val, Met, Ile or Phe
  MOD_RES (14)..(14): Gly, His or Lys
  MOD_RES (15)..(15): Trp or Met
  MOD_RES (16)..(16): Gln, Leu, His, Glu, Asn, Asp, Ser or Gly
  MOD_RES (17)..(17): Asp, Asn, Ser, Lys, Arg, Glu or His
  MOD_RES (18)..(18): Trp or Ser
  MOD_RES (19)..(20): Ile or Val
  MOD_RES (21)..(21): Ala, Ser, Tyr or Trp
  MOD_RES (23)..(23): Glu, Lys, Gln, Met, Pro, Leu, Arg, His or Lys
  MOD_RES (24)..(24): Gly, Glu, Asp, Lys, Ser, Gln, Arg or Phe
  MOD_RES (25)..(25): Tyr or Phe
  MOD_RES (26)..(26): Ala, Ser, Gly, Met, Gln, His, Glu, Asp, Leu, Asn, Lys or Tyr
  MOD_RES (27)..(27): Ala or Pro
  MOD_RES (28)..(28): Tyr, Phe, Asn, Ala or Arg
  MOD_RES (29)..(29): Tyr, His, Glu, Phe or Arg
  MOD_RES (31)..(31): Glu, Asp, Ala, Ser, Tyr, His, Lys, Arg, Gln or Gly
  MOD_RES (33)..(33): Glu, Asp, Leu, Val, Lys, Gly, Thr, Ala or Gln
  MOD_RES (35)..(35): Ala, Ser, Ile, Asn, Pro, Glu, Asp, Phe, Gln or Leu
  MOD_RES (36)..(36): Phe, Tyr, Leu, Asn, Gly or Arg
  MOD_RES (37)..(37): Pro, Ser, Ala or Val
  MOD_RES (38)..(38): Leu, Met, Glu, Phe or Val
  MOD_RES (39)..(39): Asn, Asp, Thr, Gly, Ala, Arg, Leu or Pro
  MOD_RES (40)..(40): Ser, Ala, Glu, Asp, Thr, Leu, Lys, Gln or His
  MOD_RES (41)..(41): Tyr, His, Cys, Ile, Arg, Asp, Asn, Lys, Ser, Glu or Gly
  MOD_RES (42)..(42): Met, Leu, Phe, Val, Gly or Tyr
  MOD_RES (43)..(43): Asn, Glu, Thr, Pro, Lys, His, Gly, Met, Val or Arg
  MOD_RES (44)..(44): Ala, Ser, Gly, Pro or Phe
  MOD_RES (45)..(45): Thr, Ser, Leu, Pro, His or Met
  MOD_RES (46)..(46): Asn, Lys, Val, Thr or Gln
  MOD_RES (47)..(47): His, Tyr or Lys
  MOD_RES (48)..(48): Ala, Thr, Leu or Tyr
  MOD_RES (49)..(49): Ile, Thr, Val, Phe, Tyr, Met or Pro
  MOD_RES (50)..(50): Val, Leu, Met, Ile or His
  MOD_RES (51)..(51): Gln, Arg or Thr
  MOD_RES (52)..(52): Thr, Ser, Ala, Asn or His
  MOD_RES (53)..(53): Leu, Asn or Ile
  MOD_RES (54)..(54): Val, Met, Leu, Pro or Ile
  MOD_RES (55)..(55): His, Asn, Arg, Lys, Tyr or Gln
  MOD_RES (56)..(56): Phe, Leu, Ser, Asn, Met, Ala, Arg, Glu, Gly or Gln
  MOD_RES (57)..(57): Ile, Met, Leu, Val, Lys, Gln, Ala or Tyr
  MOD_RES (58)..(58): Asn, Phe, Lys, Glu, Asp, Ala, Gln, Gly, Leu or Val
  MOD_RES (59)..(59): Pro, Asn, Ser, Val or Asp
  MOD_RES (60)..(60): Glu, Asp, Asn, Lys, Arg, Ser, Gly, Thr, Gln, Pro or His
  MOD_RES (61)..(61): Thr, His, Tyr, Ala, Ile, Lys, Asp, Ser, Gly or Arg
  MOD_RES (62)..(62): Val, Ile, Thr, Ala, Leu or Ser
  MOD_RES (63)..(63): Pro, Gly, Ser, Asp or Ala
  MOD_RES (64)..(64): Lys, Leu, Pro, Ala, Ser, Glu, Arg or Gly
  MOD_RES (65)..(65): Pro, Ala, Val, Thr or Ser
  MOD_RES (66)..(66): Cys, Val or Ser
  MOD_RES (68)..(68): Ala, Val or Thr
  MOD_RES (70)..(70): Thr, Ala, Glu, Val, Gly, Asp or Tyr
  MOD_RES (71)..(71): Gln, Lys, Glu, Arg or Val
  MOD_RES (72)..(72): Leu, Met, Thr or Tyr
  MOD_RES (73)..(73): Asn, Ser, Gly, Thr, Asp, Glu, Lys or Val
  MOD_RES (74)..(74): Ala, Pro, Gly or Ser
  MOD_RES (75)..(75): Ile, Thr, Leu or Val
  MOD_RES (76)..(76): Ser, Pro, Ala, Thr, Asn or Gly
  MOD_RES (77)..(77): Val, Ile, Leu or Met
  MOD_RES (79)..(79): Tyr, Phe, Arg, Thr, Tyr or Met
  MOD_RES (80)..(80): Phe, Tyr, His, Leu, Ile, Lys, Gln or Val
  MOD_RES (81)..(81): Asp, Leu, Asn or Glu
  MOD_RES (82)..(82): Asp, Ser, Arg, Asn, Glu, Ala, Lys, Gly or Pro
  MOD_RES (83)..(83): Ser, Asn, Asp, Tyr, Ala, Gly, Gln, Met, Glu, Asn or Lys
  MOD_RES (84)..(84): Ser, Asn, Glu, Asp, Val, Lys, Gly, Gln or Arg
  MOD_RES (85)..(85): Asn, Lys, Thr, Pro, Val, Ile, Arg, Ser or Gln
  MOD_RES (86)..(86): Val, Ile, Thr or Ala
  MOD_RES (87)..(87): Ile, Asn, Val, Leu, Tyr, Asp or Ala
  MOD_RES (88)..(88): Leu, Tyr, Lys or Ile
  MOD_RES (89)..(89): Lys, Arg, Asn, Tyr, Phe, Thr, Glu or Gly
  MOD_RES (90)..(90): Lys, Arg, His, Gln, Asn, Glu or Val
  MOD_RES (91)..(91): Tyr, His, Glu or Ile
  MOD_RES (92)..(92): Arg, Glu, Gln, Pro or Lys
  MOD_RES (93)..(93): Asn, Asp, Ala, Glu, Gly or Lys
  MOD_RES (94)..(94): Met or Ala
  MOD_RES (95)..(95): Val, Ile, Ala, Thr, Ser or Lys
  MOD_RES (96)..(96): Val or Ala
  MOD_RES (97)..(97): Arg, Lys, Gln, Asp, Glu, Val, Ala, Ser or Thr
  MOD_RES (98)..(98): Ala, Ser, Glu, Gly, Arg or Thr
  MOD_RES (100)..(100): Gly, Ala or Thr
  MOD_RES (102)..(102): His, Arg, Gly, Leu or Ser
CXXXXXXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 9 - protein, 5 aa, Artificial Sequence
  Description of Artificial Sequence Synthetic polypeptide
  MOD_RES (2)..(5): Variable amino acid
CXXXX

SEQ ID NO: 10 - DNA, 24 bp, Artificial Sequence
  Description of Artificial Sequence Synthetic probe
gccgtgcgtacttagacatgcatg

SEQ ID NO: 11 - DNA, 1822 bp, Homo sapiens
  CDS (49)..(1341)
ggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatgaaaaaaaaaaaaaaaaaa

SEQ ID NO: 12 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 13 - protein, 35 aa, Artificial Sequence
  Description of Artificial Sequence Synthetic polypeptide
LYVDFSDVGWDDWIVAPPGYQAFYCHGECPFPLAD

SEQ ID NO: 14 - protein, 35 aa, Artificial Sequence
  Description of Artificial Sequence Synthetic polypeptide
LYVDFSDVGWNDWIVAPPGYHAFYCHGECPFPLAD

SEQ ID NO: 15 - protein, 33 aa, Artificial Sequence
  Description of Artificial Sequence Synthetic polypeptide
HFNSTNHAVVQTLVNSVNSKIPKACCVPTELSA

SEQ ID NO: 16 - protein, 33 aa, Artificial Sequence
  Description of Artificial Sequence Synthetic polypeptide
HLNSTNHAVVQTLVNSVNSKIPKACCVPTELSA

SEQ ID NO: 17 - protein, 28 aa, Artificial Sequence
  Description of Artificial Sequence Synthetic polypeptide
ISMLYLDENEKVVLKYNQEMVVEGCGCR

SEQ ID NO: 18 - protein, 28 aa, Artificial Sequence
  Description of Artificial Sequence Synthetic polypeptide
ISMLYLDENEKVVLKYNQEMVVEGCGCR